(19) United States
(12) Patent Application Publication
(10) Pub. No.: US 2024/0239892 A1
(43) Pub. Date: Jul. 18, 2024

(54) ANTI-PD-1 POLYPEPTIDES AND THEIR USE

(71) Applicants: SUZHOU KANOVA BIOPHARMACEUTICAL CO., LTD., Suzhou (CN); BEIJING KANOVA BIOPHARMACEUTICAL CO., LTD., Beijing (CN)

(72) Inventors: Li GUO, Beijing (CN); Jiaming LI, Beijing (CN); Xiaoxu QI, Beijing (CN); Chen DONG, Beijing (CN)

(73) Assignees: SUZHOU KANOVA BIOPHARMACEUTICAL CO., LTD., Suzhou (CN); BEIJING KANOVA BIOPHARMACEUTICAL CO., LTD., Beijing (CN)

(21) Appl. No.: 18/562,298

(22) PCT Filed: May 18, 2022
(86) PCT No.: PCT/CN2022/093456

§ 371 Date: Nov. 18, 2023

(30) Foreign Application Priority Data
May 18, 2021 (WO) PCT/CN2021/094422

(51) Int. Cl. C07K 16/28; A61K 47/68; A61K 51/10; A61P 35/00; A61K 39/00

(52) U.S. Cl. CPC C07K 16/2818; A61K 47/6849; A61K 51/1027; A61P 35/00; A61K 2039/505; C07K 2317/21; C07K 2317/24; C07K 2317/31

(57) ABSTRACT

This present invention provides anti-PD-1 polypeptides or fragments thereof. Further provided are methods of using the antibody or fragments thereof to treat and diagnose diseases such as cancer, infection or immune disorders.

The present invention is related to anti-PD-1 polypeptides including anti-PD-1 antibodies and their immunoreactive fragments, an isolated nucleotide encoding the anti-PD-1 antibodies or their immunoreactive fragments, particularly in the treatment of a medical disorder in which the pathogenic cells use PD-1/PD-L1 checkpoint for immune escape. The invention particularly concerns humanized anti-PD-1 antibodies and their antigen binding fragments capable of enhancing the activation of the immune system against diseased tissues including cancerous cells and infected cells expressing PD-L1 and/or PD-L2.

### BACKGROUND OF THE INVENTION

The cDNA of programmed cell death 1 (PD-1) was isolated in 1992 from murine T cell hybridomas and hematopoietic progenitor cell lines undergoing apoptosis. Researches shown that PD-1 deficiency leads to different autoimmune phenotypes in various mouse strains. PD-1-deficient allogeneic T cells with transgenic T cell receptor (TCR) exhibit enhanced response to alloantigens, which indicates that PD-1 on T cells plays a negative regulatory role in response to antigens.

Several studies have facilitated the discovery of molecules that interact with PD-1. In 1999, B7 homolog 1 (B7-H1, also known as programmed death ligand 1 [PD-L1]) of PD-1 was identified, and was shown to be an inhibitor of human T cell response in vitro. Later B7-H1 (hereinafter referred to as PD-L1) was proved to be a binding and functional partner of PD-1. Next, it was determined that PD-L1 deficient mice (PD-L1 KO mice) are prone to autoimmune diseases, although these mice do not spontaneously develop the disease. It later became clear that the interaction of PD-L1/PD-1 plays an important role in the suppression of T cell responses in vivo, especially in the tumor microenvironment.

In addition, studies also show that tumor-associated PD-L1 promotes apoptosis of activated T cells (Dong H. et al. Tumor-associated B7-H1 promotes T-cell apoptosis: a potential mechanism of immune evasion. Nature medicine. 2002; 8(8):793-800), and stimulates the production of IL-10 in human peripheral blood T cells (Dong H, et al., B7-H1, a third member of the B7 family, co-stimulates T-cell proliferation and interleukin-10 secretion. Nature medicine. 1999; 5(12):1365-9) to mediate immune suppression. It's also well known that the effect of PD-L1 on immunosuppression is more complicated. In addition to T cell apoptosis and IL-10 induction, PD-L1 can also induce T cell dysfunction through a variety of mechanisms. The PD-1 pathway has also been shown to promote T cell disability in vitro and in vivo.

In recent years, FDA approved two PD-1 mAbs (PD-1 monoclonal antibodies) for the treatment of human cancer, one of which comes from Bristol-Myers Squibb (Opdivo, nivolumab, MDX-1106, BMS-936558, ONO-4538), and the other from Merck (Keytruda, Pembrolizumab, lambrolizumab, MK-3475). In addition, in hundreds of clinical trials involving thousands of patients, multiple monoclonal antibodies against PD-1 or PD-L1 are being actively developed. To date, anti-PD therapy has brought significant clinical benefits by inducing regression of advanced and metastatic tumors and improving survival rates. More importantly, anti-PD therapy has long-lasting effects, tolerable toxicity, and has been shown to be applicable to a wide range of cancer types, especially in solid tumors. Due to its non-overlapping mechanism relative to other cancer therapies, anti-PD therapy is being combined with almost all cancer treatment methods in an attempt to further expand the therapeutic effect. In addition to combining with various cancer immunotherapies (such as cancer vaccines, costimulatory and co-suppressive antibodies, and adoptive cell therapy), various clinical trials have also started to combine anti-PD-1 therapies with chemotherapy, radiotherapy and targeted therapies.

Although antibodies against PD-1 are developed, there are still spaces for improvement for an antibody against PD-1 as a therapeutic agent. Accordingly, there is desire in the art to develop novel anti-PD-1 antibodies with higher specificity and efficiency.

### SUMMARY OF THE INVENTION

Provided herein are an antibody and its immunoreactive fragments binding to PD-1 molecule expressed on cells (for example, cancer cells) with high affinity and facilitating effective immune response against cancer cells. The antibody and its immunoreactive fragments provided herein are capable of enhancing the activation of the immune system, and thus providing important therapeutic and diagnostic agents for use in targeting pathological conditions associated with expression and/or activity of PD-1 molecule. In one aspect, the present invention provides an isolated antibody or antigen binding fragment thereof, comprising a heavy chain (HC) variable region sequence and a light chain (LC) variable region sequence, wherein the antibody binds to an extracellular domain of PD-1 with a binding affinity of better than 10 nM or about 10 nM, better than 8 nM or about 8 nM, better than 6 nM or about 6 nM, better than 4 nM or about 4 nM, better than 2 nM or about 2 nM, better than 1 nM or about nM, as determined by SPR analysis, for example, about 0.5-4 nM, about 0.8-4.0 nM, about 1.0-4.0 nM, about 2.0-4.0 nM, about 3.0-4.0 nM, about 0.6-3.5 nM, about 1.4-3.5 nM, about 2.5-3.5 nM, about 0.7-2.5 nM, about 0.8-2.0 nM, about 1.0-2.0 nM, about 0.4 nM, 0.3 nM, 0.2 nM, 0.1 nM, or better, as determined by SPR analysis.

In certain embodiments, the present invention provides an antibody or antigen binding fragment thereof, comprising at least one of the following:
(a) CDR1H sequence comprising the amino acid sequence as shown by GFTFSSYGMS (SEQ ID NO: 1),
(b) CDR2H sequence comprising the amino acid sequence as shown by IISGGGRDIYYLDSVKG (SEQ ID NO: 2),
(c) CDR3H sequence comprising the amino acid sequence as shown by PIYDAYSFAY (SEQ ID NO: 3),
(d) CDR1L sequence comprising the amino acid sequence as shown by RASQTISNNLH (SEQ ID NO: 4),
(e) CDR2L sequence comprising the amino acid sequence as shown by YASQSIS (SEQ ID NO: 5), and
(f) CDR3L sequence comprising the amino acid sequence as shown by QQSYSWPLT (SEQ ID NO: 6).

In certain embodiments, the present invention provides an antibody or antigen binding fragment thereof, wherein
(a) the HC comprises
CDR1H sequence comprising the amino acid sequence as shown by GFTFSSYGMS (SEQ ID NO: 1),
CDR2H sequence comprising the amino acid sequence as shown by IISGGGRDIYYLDSVKG (SEQ ID NO: 2), and
CDR3H sequence comprising the amino acid sequence as shown by PIYDAYSFAY (SEQ ID NO: 3),
(b) the LC comprises
CDR1L sequence comprising the amino acid sequence as shown by RASQTISNNLH (SEQ ID NO: 4),
CDR2L sequence comprising the amino acid sequence as shown by YASQSIS (SEQ ID NO: 5), and
CDR3L sequence comprising the amino acid sequence as shown by QQSYSWPLT (SEQ ID NO: 6).

The CDR sequences are determined according to Kabat et al, Sequences of Proteins of Immunological Interest, Fifth Edition, NIH Publication 91-3242, Bethesda MD (1991), vols. 1-3.

In certain embodiments, the antibody is a chimeric antibody, humanized antibody, or human antibody. In certain embodiments, the antibody or antigen binding fragment thereof of the present invention further comprises a human acceptor framework. In certain embodiments, the human acceptor framework is derived from a human immunoglobulin framework or a human consensus framework. In certain embodiments, the human acceptor framework comprises subgroup kappa I framework sequences for VL, and subgroup III framework sequences for VH. The subgroup of sequences is a subgroup as in Kabat et al., Sequences of Proteins of Immunological Interest, Fifth Edition, NIH Publication 91-3242, Bethesda MD (1991), vols. 1-3. In certain embodiments, for the VL, the subgroup is subgroup kappa I as in Kabat et al, supra. In certain embodiments, for the VH, the subgroup is subgroup III as in Kabat et al, supra.

In certain embodiments, the antibody or antigen binding fragment thereof comprises human consensus framework. In some embodiments, the antibody or antigen binding fragment thereof comprises human consensus framework with amino acid sequence changes, for example, 1-15, 1-10, 2-9, 3-8, 4-7 or 5-6 amino acid changes.

In certain embodiments, the antibody or antigen binding fragment thereof of the invention comprises the HC variable region sequence comprising an amino acid sequence as shown by SEQ ID NO: 7 or 8, or an amino acid sequence having more than 80%, 85%, 90%, 91%, 92%, 93%, 94%, 95%, 96%, 97%, 98% or 99% identity with SEQ ID NO: 7 or 8. In certain embodiments, the antibody or antigen binding fragment thereof of the invention comprises the LC variable region sequence comprising an amino acid sequence as shown by SEQ ID NO: 9 or SEQ ID NO: 10, or an amino acid sequence having more than 80%, 85%, 90%, 91%, 92%, 93%, 94%, 95%, 96%, 97%, 98% or 99% identity with SEQ ID NO: 9 or 10.

In certain embodiments, the HC variable region sequence comprises an amino acid sequence as shown by SEQ ID NO: 7 and the LC variable region sequence comprises an amino acid sequence as shown by SEQ ID NO: 9 or SEQ ID NO: 10. In certain embodiments, the HC variable region sequence comprises an amino acid sequence as shown by SEQ ID NO: 8 and the LC variable region sequence comprises an amino acid sequence as shown by SEQ ID NO: 9 or SEQ ID NO: 10. In certain embodiments, the antibody or antigen binding fragment thereof of the invention comprises the HC sequence comprising an amino acid sequence as shown by SEQ ID NO: 11 or 12, or an amino acid sequence having more than 80%, 85%, 90%, 91%, 92%, 93%, 94%, 95%, 96%, 97%, 98% or 99% identity with SEQ ID NO: 11 or 12. In certain embodiments, the antibody or antigen binding fragment thereof of the invention comprises the LC sequence comprising an amino acid sequence as shown by SEQ ID NO: 13 or SEQ ID NO: 14, or an amino acid sequence having more than 80%, 85%, 90%, 91%, 92%, 93%, 94%, 95%, 96%, 97%, 98% or 99% identity with SEQ ID NO: 13 or 14. In certain embodiments, the HC sequence comprises an amino acid sequence as shown by SEQ ID NO: 11 and the LC sequence comprises an amino acid sequence as shown by SEQ ID NO: 13 or SEQ ID NO: 14. In certain embodiments, the HC sequence comprises an amino acid sequence as shown by SEQ ID NO: 12 and the LC variable region sequence comprises an amino acid sequence as shown by SEQ ID NO: 13 or SEQ ID NO: 14. In certain embodiments, the antibody is an IgG1, IgG2 or IgG4 isotype. In certain embodiments, the antigen binding fragment is any one selected from the group consisting of Fab, F(ab′)2, Fab′, scFv, and Fv. In certain embodiments, the antibody or antigen binding fragment thereof of the invention is a blocking antibody or an antagonist antibody which inhibits or reduces biological activity of the PD-1 molecule it binds. Preferred the blocking antibodies or antagonist antibodies substantially or completely inhibit the biological activity of the PD-1 molecule.

In one aspect, the present invention provides a bispecific antibody comprising the antibody or antigen binding fragment thereof of the present invention and a second antibody or antigen binding fragment thereof. In certain embodiments, the second antibody or antigen binding fragment thereof specifically binds to a tumor antigen expressed on the surface of a tumor cell, wherein the tumor antigen is selected from the group consisting of A33; ADAM-9; ALCAM; BAGE; beta-catenin; CA125; Carboxypeptidase M; CD103; CD19; CD20; CD22; CD23; CD25; CD27; CD28; CD36; CD40/CD154; CD45; CD46; CD5; CD56; CD79a/CD79b; CDK4; CEA; CTLA4; Cytokeratin 8; EGF-R; EphA2; ErbB1; ErbB3; ErbB4; GAGE-1; GAGE-2; GD2/GD3/GM2; HER-2/neu; human papillomavirus-E6; human papillomavirus-E7; JAM-3; KID3; KID31; KSA (17-1A); LUCA-2; MAGE-1; MAGE-3; MART; MUC-1; MUM-1; N-acetylglucosaminyltransferase; Oncostatin M; p15; PIPA; PSA; PSMA; ROR1; TNF-β receptor; TNF-a receptor; TNF-γ receptor; Transferrin Receptor; and VEGF receptor. In some embodiments, the second antibody or antigen binding fragment thereof specifically binds to a checkpoint protein expressed on the surface of an abnormal cell or an immune cell, wherein the immune checkpoint protein is selected from the group consisting of 2B4; 4-1BB; 4-1BB ligand, B7-1; B7-2; B7H2; B7H3; B7H4; B7H6; BTLA; CD155; CD160; CD19; CD200; CD27; CD27 ligand; CD28; CD40; CD40 ligand; CD47; CD 48; CTLA-4; DNAM-1; Galectin-9; GITR; GITR ligand; HVEM; ICOS; ICOS ligand; IDOI; KIR; 3DL3; LAG-3; OX40; OX40 ligand; PD-L1; PD-1; PD-L2; LAG3; PGK; SIRP α; TIM-3; TIGIT; VSIG8.

In one aspect, the present invention provides a polypeptide comprising the antibody or antigen binding fragment thereof of the present invention.

In one aspect, the present invention provides a polypeptide comprising the HC variable region and/or LC variable region of the antibody or antigen binding fragment thereof of the present invention.

In one aspect, the present invention provides a conjugate comprising the antibody or antigen binding fragment thereof of the present invention. In certain embodiments, the present invention provides a conjugate comprising the antibody or antigen binding fragment thereof of the present invention, linked to a therapeutic agent. In certain embodiments, the therapeutic agent is a cytotoxin or a radioactive isotope.

In one aspect, the present invention provides a composition comprising the antibody or antigen binding fragment thereof, the bispecific antibody, the polypeptide, the conjugate of the present invention, and a pharmaceutically acceptable carrier. In certain embodiments, the composition further comprises an anti-cancer agent. In certain embodiments, the agent is an antibody, a chemotherapeutic agent, a radiation therapeutic agent, a hormonal therapeutic agent, a toxin or an immunotherapeutic agent. In certain embodiments, the composition further comprises antibodies or agents inhibiting checkpoints.

In one aspect, the present invention provides an article of manufacture or kit for treating cancer, comprising the antibody or antigen binding fragment thereof, the bispecific antibody, the polypeptide, the conjugate or the composition of the present invention, and package insert with necessary information about the use of the antibody or antigen binding fragment thereof, the bispecific antibody, the polypeptide, the conjugate or the composition of the present invention.

In one aspect, the present invention provides an article of manufacture or kit for diagnosing cancer or determining the presence and/or the amount of PD-1, comprising the antibody or antigen binding fragment thereof of the present invention, and package insert with necessary information about the use of the antibody or antigen binding fragment thereof of the present invention.

In one aspect, the present invention provides an isolated nucleic acid encoding the antibody or antigen binding fragment thereof of the present invention. In certain embodiments, the present invention provides an isolated nucleic acid encoding the HC variable region and/or LC variable region of the antibody or antigen binding fragment thereof of the present invention. In certain embodiments, the present invention provides an expression vector comprising the nucleic acid, or a host cell comprising the expression vector.

In one aspect, the present invention provides a method for preparing an antibody or antigen binding fragment thereof comprising expressing the antibody or antigen binding fragment thereof in the host cell stated above and isolating the antibody or antigen binding fragment thereof from the host cell.

In one aspect, the present invention provides a method for treatment of a cancer, comprising administrating an effective amount of the antibody or antigen binding fragment thereof, the bispecific antibody, the polypeptide, the conjugate, the composition, the article of manufacture or kit of the present invention, described above, to a subject having the cancer disease. In certain embodiments, the cancer is any one selected from the group consisting of: lymphoma, melanoma, colorectal adenocarcinoma, prostate cancer, breast cancer, colon cancer, lung cancer, liver cancer, gastric cancer, and renal clear cell carcinoma.

In one embodiment, an effective amount of the antibody or antigen binding fragment thereof, the bispecific antibody, the polypeptide, the conjugate, the composition, the article of manufacture or kit of the present invention, described above, is the sole therapeutic anti-cancer agent administered to the patient. In another embodiment, they are administered in combination with another antibody or antibody fragment or anti-cancer agent that includes, but is not limited to, an antibody against a checkpoint molecule or its receptor (for example, anti-CTLA-4 antibody, anti-B7S1 antibody, anti-PD-L1 antibody, anti-B7H3 antibody, etc.); an anti-epidermal growth factor receptor (EGFR) agent such as, e.g., panitumumab, the anti-EGFR antibody cetuximab (Erbitux®), and the EGFR tyrosine kinase (TK) inhibitors gefitinib (Iressa®) and erlotinib (Tarceva®); an alkylating agent such as, e.g., cisplatin, carboplatin, oxaliplatin, nedaplatin, satraplatin, triplatin tetranitrate, mechlorethamine, cyclophosphamide, chlorambucil and ifosfamide; paclitaxel and docetaxel; and topoisomerase inhibitors such as, e.g., irinotecan, topotecan, amsacrine, etoposide, etoposide phosphate and teniposide.

In certain embodiments, the antibody or antigen binding fragment thereof, the bispecific antibody, the polypeptide, the conjugate, the composition, the article of manufacture or kit of the present invention, described above, is administered in combination with another anti-PD-1 antibody or an anti-PD-L1 antibody to achieve synergistic effect on the treatment of cancer.

In one aspect, the present invention provides a method for treatment of a cancer, comprising administrating an effective amount of the antibody or antigen binding fragment thereof, the bispecific antibody, the polypeptide, the conjugate, the composition, the article of manufacture or kit of the present invention, described above to a subject having the cancer disease. In certain embodiments, the cancer is any one selected from the group consisting of: prostate cancer, breast cancer, colon cancer, lung cancer, liver cancer, gastric cancer, and renal clear cell carcinoma, bladder cancer, breast cancer, colorectal cancer, endometrial cancer, esophageal cancer, head and neck cancer, kidney cancer, leukemia, liver cancer, lung cancer, lymphoma, melanoma, pancreatic cancer, prostate cancer, and thyroid cancer. In certain embodiments, the cancer is any one selected from the group consisting of: microsatellite instability-high colorectal cancer, a microsatellite stable colorectal cancer, a triple negative breast cancer, a merkel cell carcinoma, an endometrial cancer, or an esophageal cancer.

In one aspect, the present invention provides a method for treatment of a cancer, comprising: a) treating activated T cells, B cells, NK cells, dendritic cells, monocytes or the combination of any of them, or PBMCs in vitro with the antibody or antigen binding fragment thereof, the bispecific antibody, the polypeptide, the conjugate, the composition, the article of manufacture or kit of the present invention described above; and b) administering the treated T cells, B cells, NK cells, dendritic cells, monocytes or the combination of any of them, or PBMCs to the patient. In some embodiments, the method further comprises, prior to step a), isolating the T cells, B cells, NK cells, dendritic cells or monocytes from an individual. In some embodiments, the T cells and/or NK cells are from the patient to be treated. In some embodiments, the T cell is a tumor-infiltrating T lymphocyte, a CD4+ T cell, a CD8+ T cell, or the combination thereof.

Thus, in on aspect, the present invention also provides group of lymphocytes, comprising T cells and/or NK cells derived from a subject and are treated in vitro with the antibody or antigen binding fragment thereof, the bispecific antibody, the polypeptide, the conjugate, the composition, the article of manufacture or kit of the present invention described above. In some embodiments, the T cells and/or NK cells are from the patient to be treated. In some embodiments, the T cell is a tumor-infiltrating T lymphocyte, a CD4+ T cell, a CD8+ T cell, or the combination thereof.

In one aspect, the present invention provides a method for treating or inhibiting infection in a patient in need thereof, comprising administering an effective amount of the antibody or antigen binding fragment thereof, the bispecific antibody, the polypeptide, the conjugate, the composition, the article of manufacture or kit of the present invention described above to the patient. In some embodiments, the infection is viral, bacterial, fungal, or parasite infection. In some certain embodiments, the infection is HIV infection.

In one aspect, the present invention provides a method for detection or quantitation of expression or activity of PD-1 polypeptide, comprising contacting the antibody or antigen binding fragment thereof of the present invention with a sample from a subject. In certain embodiments, the antibody or antigen binding fragment thereof is labeled with detectable substance. In certain embodiments, the antibody or antigen binding fragment thereof is radio-labeled, fluorescence-labeled or enzyme-labeled.

In one aspect, the present invention provides a method for predicting the risk of developing a cancer of a subject comprising detecting, quantitating, or monitoring expression or activity of PD-1 polypeptide by using the antibody or antigen binding fragment thereof of the present invention.

In one aspect, the present invention provides a method for monitoring the effectiveness of an agent to treat a cancer exhibiting elevated expression or activity of PD-1, comprising detection or quantitation of expression or activity of PD-1 polypeptide by using the antibody or antigen binding fragment thereof of the present invention.

In one embodiment, the present invention provides an isolated polynucleotide encoding a human anti-PD-1 antibody or a fragment thereof, wherein the antibody comprises an amino acid sequence selected from the group consisting of SEQ ID NOs: 7-14, preferably from the group consisting of SEQ ID NOs: 11-14. In certain embodiments, the human PD-1 antibody comprises a heavy chain and a light chain, wherein the heavy chain comprises SEQ ID NO: 7, and the light chain comprises SEQ ID NO: 9 or SEQ ID NO: 10. In certain embodiments, he human PD-1 antibody comprises a heavy chain and a light chain, wherein the heavy chain comprises SEQ ID NO: 8, and the light chain comprises SEQ ID NO: 9 or SEQ ID NO: 10. In certain embodiments, the antibody comprises a heavy chain and a light chain, wherein the heavy chain comprises SEQ ID NO: 11, and the light chain comprises SEQ ID NO: 13 or SEQ ID NO:14. In certain embodiments, the antibody comprises a heavy chain and a light chain, wherein the heavy chain comprises SEQ ID NO: 12, and the light chain comprises SEQ ID NO: 13 or SEQ ID NO:14. More preferably, the antibody comprises a heavy chain and a light chain, wherein the light chain comprises SEQ ID NO: 10 or SEQ ID NO: 14, and the heavy chain comprises SEQ ID NO: 7, SEQ ID NO:8, SEQ ID NO:11 or SEQ ID NO:12. In some embodiments, the antibody or antibody fragment comprises VH and VL domains of a single chain antibody fragment. In some embodiments, the VH domain comprises a sequence selected from the group consisting of SEQ ID NO: 1, SEQ ID NO: 2, and SEQ ID NO: 3. In some embodiments the VH domain comprises three CDRs, wherein each of the three CDRs comprises a sequence selected from the group consisting of SEQ ID NO: 1, SEQ ID NO: 2, and SEQ ID NO: 3. In some embodiments, the VH domain comprises SEQ ID NO: 7 or SEQ ID NO: 8. In some embodiments, the VL domain comprises a sequence selected from the group consisting of SEQ ID NO: 4, SEQ ID NO: 5, and SEQ ID NO: 6. In some embodiment the VL domain comprises three CDRs, wherein each of the three CDRs comprises a sequence selected from the group consisting of SEQ ID NO: 4, SEQ ID NO: 5, and SEQ ID NO: 6. In some embodiments, the VL domain comprises SEQ ID NO: 9 or SEQ ID NO: 10.

In one embodiment, the present invention provides a method for diagnosing a disease, disorder or condition associated with the expression of PD-1 on a cell, or determining the presence and/or the amount of PD-1, wherein the method comprises a) contacting the cell with a human anti-PD-1 antibody or fragment thereof, wherein the antibody or a fragment thereof comprises an amino acid sequence selected from the group consisting of SEQ ID NOs: 7-14; and b) detecting the presence of PD-1 wherein the presence of PD-1 diagnoses for the disease, disorder or condition associated with the expression of PD-1. In certain embodiments, the disease, disorder or condition associated with the expression of PD-1 is cancer.

In one embodiment, the present invention provides a method of diagnosing, prognosing, or determining risk of a PD-1-related disease in a mammal, wherein the method comprises detecting the expression of PD-1 in a sample derived from the mammal comprising: a) contacting the sample with a human anti-PD-1 antibody or fragment thereof, wherein the antibody or a fragment thereof comprises an amino acid sequence selected from the group consisting of SEQ ID NOs: 7-14; and b) detecting the presence of PD-1 wherein the presence of PD-1 diagnoses for a PD-1 related disease in the mammal. In certain embodiments, the PD-1-related disease is cancer.

In one embodiment, the present invention provides a method of blocking PD-1-dependent T cells, B cells, NK cells, dendritic cells or monocytes inhibition, wherein the method comprises contacting a cell with a human anti-PD-1 antibody or fragment thereof, wherein the antibody or a fragment thereof comprises an amino acid sequence selected from the group consisting of SEQ ID NOs: 7-14. In some embodiments, the cell is selected from the group consisting of B cells, T cells or NK cells. In one embodiment, the present disclosure provides a method of blocking PD-1-dependent immunosuppression in a mammal, wherein the method comprises administering to the mammal an effective amount of an anti-PD-1 antibody or fragment thereof, described above. In certain embodiments, the mammal comprises cells selected from the group consisting of a PD-1-expressing T cells, B cells, NK cells, dendritic cells or monocytes and PD-L1 and/or PD-L2-expressing abnormal cells.

In one embodiment, the present invention provides a method of providing an anti-tumor immunity in a mammal, wherein the method comprises administering to the mammal an effective amount of a genetically modified cell encoding and expressing an anti-PD-1 antibody or a fragment thereof, wherein the anti-PD-1 antibody or a fragment thereof comprises an amino acid sequence selected from the group consisting of SEQ ID NOs: 7-14.

### DRAWINGS

FIG. 1. SDS-PAGE analysis results for purified antibodies.

FIG. 2A-2B. ELISA results determining the binding affinity of antibodies to human (2A) and cynomolgus (2B) PD-1.

FIG. 3. Flow cytometry analysis of binding affinity of antibodies to Human PD-1-expressing Jurkat cells.

FIG. 4. Showing the interaction of PD-1 and PD-L1 are efficiently blocked by humanized anti-PD-1 antibodies in cell-based assay.

FIG. 5A-5C. Showing the enhancement of IL-2 (5A), IFN-γ (5B) and TNF-α (5C) secretion by human PBMCs interacted with anti-PD-1 antibodies in cytokine release assays.

FIG. 6A-6C. Showing tumor growth inhibition by antibody VH7+VL6 in vivo. FIG. 6A is the analysis results of tumor size in each mice group. FIGS. 6B and 6C separately shows the change of tumor size in each mouse.

### DETAILED DESCRIPTION OF THE INVENTION

The present invention herein provides antibodies and fragments thereof binding to PD-1 protein, especially human PD-1 protein or polypeptide. The present invention is also related to the use of the antibodies and fragments thereof for enhancing the activation of the immune system against for example cancer cells.

The invention further provides methods of making anti-PD-1 antibodies, polynucleotides encoding anti-PD-1 antibodies, and cells comprising polynucleotides encoding anti-PD-1 antibodies.

### 1. Definition

It is to be understood that the present invention is not limited to the aspects described herein, as such may, of course, vary. It is also to be understood that the terminology used herein is for describing particular aspects only, and is not intended to be limiting, since the scope of the present invention will be limited only by the appended claims.

Unless defined otherwise, all technical and scientific terms used herein have the same meanings as commonly understood by one of ordinary skill in the art to which this technology belongs. All technical and patent publications cited herein are incorporated herein by reference in their entirety. Those skilled in the art will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3rd edition; Harlow and Lane eds. (1999) Antibodies, A Laboratory Manual. MONOCLONAL ANTIBODIES: A PRACTICAL APPROACH (Shepherd, P. et al. Eds., 2000) Oxford University Press, USA, New York N.Y.

The term “PD-1”, as used herein, refers programmed cell death protein, which belongs to the superfamily of immunoglobulin and functions as co-inhibitory receptor to negatively regulate the immune system. PD-1 is a member of the CD28/CTLA-4 family, and has two known ligands including PD-L1 and PD-L2. Alternative names or synonyms for PD-1 include PDCD1, PD1, CD279 and SLEB2, et al. Representative amino acid sequence of human PD-1 is disclosed under the NCBI accession number: NP 005009.2, and the representative nucleic acid sequence encoding the human PD-1 is shown under the NCBI accession number: NM 005018.2. PD-1 proteins are expressed by circulating lymphocytes (e.g. T cells, B cells, monocytes, natural killer T cells (NK cells), and macrophages) and is a marker of activation and exhaustion.

The term “PD-L1”, as used herein, refers to programmed cell death ligand 1 (PD-L1, see, for example, Freeman et al. (2000) J. Exp. Med. 192: 1027). Alternative names or synonyms for PD-L1 include PDCDIL1, PDL1, B7H1, CD274 and B7-H, et al. Representative amino acid sequence of human PD-L1 is disclosed under the NCBI accession number: NP 054862.1, and the representative nucleic acid sequence encoding the human PD-L1 is shown under the NCBI accession number: NM 014143.3. PD-L1 is expressed in placenta, spleen, lymph nodes, thymus, heart, fetal liver, and is also found on many tumor or cancer cells. PD-L1 binds to its receptor PD-1 or B7-1, which is expressed on activated T cells, B cells and myeloid cells. The binding of PD-L1 and its receptor induces signal transduction to suppress TCR-mediated activation of cytokine production and T cell proliferation. Accordingly, PD-L1 plays a major role in suppressing immune system during particular events such as pregnancy, autoimmune diseases, tissue allografts, and is believed to allow tumor or cancer cells to circumvent the immunological checkpoint and evade the immune response. PD-L1 is also reported to be highly expressed on inflammatory macrophages as compared with resident peritoneal macrophages but can be induced on resident macrophages by classical activation stimuli such as lipopolysaccharide, IFN-γ, and polyinosinic-polycytidylic acid.

The term “PD-L2”, as used herein, refers to programmed cell death ligand 2. Alternative names or synonyms for PD-L2 include PDCDIL2, PDL2, B7-DC, Btdc and CD273, et al. Representative amino acid sequence of human PD-L2 is disclosed under the NCBI accession number: NP 079515.2.

The term “Anti-PD-1 antibody”, as used herein, refers to an antibody that is capable of specific binding to PD-1 (e.g. human, monkey or monkey PD-1). It is advantage that the Anti-PD-1 antibody specifically binds to PD-1 with an affinity which is sufficient to provide for diagnostic and/or therapeutic use. Preferably, the anti-PD-1 antibody compete with PD-L1, PD-L2 and/or the other ligands of PD-1 for binding to PD-1.

As used in the present invention, the term “antibody”, also called “immunoglobulin”, covers antibodies with structural characteristics of a native antibody and antibody-like molecules having structural characteristics different from a native antibody but exhibiting binding specificity to PD-1 molecule. The term antibody is intended to encompass immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The terms “heavy chain” (“CH”), “light chain” (“CL”), “light chain variable region” (“VL”), “heavy chain variable region” (“VH”), “framework region” (“FR”), refer to domains in naturally occurring immunoglobulins and the corresponding domains of synthetic (e.g., recombinant) binding proteins (e.g., humanized antibodies). The basic structural unit of naturally occurring immunoglobulins (e.g., IgG) is a tetramer having two light chains and two heavy chains. The amino-terminal (“N”) portion of each chain includes a variable region of about 100 to 110 or more amino acids primarily responsible for antigen recognition. The carboxy-terminal (“C” portion of each chain defines a constant region, with light chains having a single constant domain and heavy chains usually having three constant domains and a hinge region. Thus, the structure of the light chains of a naturally occurring IgG molecule is N-VL-CL-C and the structure of IgG heavy chains is N-VH-CH1-H-CH2-CH3-C (where H is the hinge region). The variable region of an IgG molecule consists of the complementarity determining regions (CDRs), which contain the residues in contact with antigen and non-CDR segments, referred to as framework segments, which maintain the structure and determine the positioning of the CDR loops. Thus, the VL and VH domains have the structure N-FR1-CDR1-FR2-CDR2-FR3-CDR3-FR4-C.

In a native antibody, the variability is not evenly distributed through the variable regions of antibodies. It is concentrated in three segments called complementarity determining regions (CDRs) or hypervariable regions both in the light chain and the heavy chain variable regions. A CDR on the heavy chain can be referred to as CDRnH, the “n” is an integer which doesn't indicate the order of the CDRs on the heavy chain. Similarly, a CDR on the light chain can be referred to as CDRnL, and the “n” is an integer labeling the CDR and doesn't indicate the order of the CDRs on the light chain. The more highly conserved portions of variable domains are called the framework (FR). The variable regions of native heavy and light chains each comprise four FR regions, connected by three CDRs. The CDRs in each chain are held together in proximity with the FR regions and, with the CDRs from the other chain, contribute to the formation of the antigen binding site of antibodies [see Kabat, E. A. et al., Sequences of Proteins of Immunological Interest National Institute of Health, Bethesda, MD (1987)]. The constant regions are not involved directly in binding an antibody to an antigen, but exhibit various effector functions, such as participation of the antibody in antibody-dependent cellular toxicity (ADCC).

The term of “antigen binding fragment” of an antibody (or simply “antibody fragment”), as used herein, refers to one or more fragments of an antibody that retain the ability to specifically bind to an antigen (e.g., PD-1 molecule, such as human PD-1). The antibody fragments comprise only a portion of an intact antibody, wherein the portion preferably retains at least one, preferably most or all, of the functions normally associated with that portion when present in an intact antibody. Examples of antibody fragments include Fab, Fab′, F(ab′)2, and Fv fragments; diabodies; linear antibodies; single-chain antibody molecules; and multispecific antibodies formed from antibody fragments.

Papain digestion of antibodies produces two identical antigen binding fragments, called “Fab” fragments, each with a single antigen binding site, and a residual Fc fragment, whose name reflects its ability to crystallize readily. The “Fab” fragment also contains the constant domain of the light chain and the first constant domain (CH1) of the heavy chain. “Fab′” fragments differ from Fab fragments by the addition of a few residues at the carboxyl terminus of the heavy chain CH1 domain including one or more cysteines from the antibody hinge region. “Fab′-SH” is the designation for Fab′ in which the cysteine residue(s) of the constant domains have a free thiol group. “F(ab′)” fragments are produced by cleavage of the disulfide bond at the hinge cysteines of the “F(ab′)2” which is pepsin digestion product.

A “Fd” fragment consists of the VH and CH1 domains. A “dAb” fragment (Ward et al., (1989) Nature 341:544-546) consists of a VH or VL domain. An isolated complementarity determining region (CDR) and a combination of two or more isolated CDRs which may optionally be joined by a synthetic linker.

A “Fv” fragment consists of the VL and VH domains of a single arm of an antibody. A single chain Fv (scFv) consists of one heavy- and one light-chain variable region covalently linked by a flexible peptide linker in one single polypeptide chain.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-48 (1993).

These antibody fragments are obtained using conventional techniques known to those with skill in the art, for example, by recombinant DNA techniques, or by enzymatic or chemical cleavage of intact immunoglobulins.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising in the monoclonal antibody preparation, such variants generally being present in minor amounts.

As used herein, the term “chimeric antibody” means an antibody in which the Fc constant region of a monoclonal antibody from one species (e.g., a mouse Fc constant region) is replaced, using recombinant DNA techniques, with an Fc constant region from an antibody of another species (e.g., a human Fc constant region). See for example, Robinson et al., PCT/US86/02269; Morrison et al., European Patent Application 173,494.

As used herein, the term “humanized antibody” refers to an antibody including a human framework region and one or more CDRs from a non-human (for example a mouse, rat, rabbit or synthetic) immunoglobulin. The non-human immunoglobulin providing the CDRs is termed a “donor,” and the human immunoglobulin providing the framework is termed an “acceptor.” In one aspect, all the CDRs are from the donor immunoglobulin in a humanized immunoglobulin. Hence, all parts of a humanized immunoglobulin, except possibly the CDRs, are substantially identical to corresponding parts of natural human immunoglobulin sequences. Humanized antibodies can be constructed by means of genetic engineering (see for example, U.S. Pat. No. 5,585,089).

An “acceptor human framework” means a framework comprising the amino acid sequence of a light chain variable domain (VL) framework or a heavy chain variable domain (VH) framework derived from a human immunoglobulin framework or a human consensus framework. An acceptor human framework “derived from” a human immunoglobulin framework or a human consensus framework may comprise the same amino acid sequence thereof, or it may contain amino acid sequence changes. In some embodiments, the number of amino acid changes is 1-10, 2-9, 3-8, 4-7 or 5-6.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In certain embodiments, for the VL, the subgroup is subgroup kappa I as in Kabat et al, supra. In certain embodiments, for the VH, the subgroup is subgroup III as in Kabat et al, supra.

The term “human antibody” as used herein, is intended to include antibodies having variable and constant regions derived from human germline immunoglobulin sequences. The human antibodies of the present technology may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a rabbit, have been grafted onto human framework sequences. Thus, as used herein, the term “human antibody” refers to an antibody in which substantially every part of the protein (e.g., CDR, framework, CL, HC domains (e.g., CH1, CH2, CH3), hinge, VL, VH) is substantially non-immunogenic in humans, with only minor sequence changes or variations. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes.

As used herein the phrase “bispecific antibody” or “bispecific antigen binding antibody” or “bifunctional antibody” is an artificial hybrid antibody having two different heavy/light chain pairs and two different binding sites. As far as the present invention is concerned, the “bispecific antibody” specifically binds to PD-1 and another antigen, for example, a tumor antigen expressed on a tumor cell.

A “conjugate” is an antibody conjugated to one or more heterologous molecule(s), including but not limited to a cytotoxic agent.

A “blocking” antibody or an “antagonist” antibody is one which inhibits or reduces biological activity of the antigen it binds. Preferred blocking antibodies or antagonist antibodies substantially or completely inhibit the biological activity of the antigen.

The term “isolated” as used herein refers to molecules or biological or cellular materials being substantially free from other materials. For example, a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

As used herein, percent of “homology” or “identity” is used in the context of two or more nucleic acids or polypeptide sequences, referring to two or more sequences or subsequences that are the same or have a specified percentage of nucleotides or amino acid residues that are the same, e.g., at least 80% identity, preferably at least 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art.

Preferably, default parameters are used for alignment. A preferred alignment program is BLAST, using default parameters. Preferred programs are BLASTN and BLASTP. Details of these programs can be found at the following Internet address: ncbi.nlm.nih.gov/cgi-bin/BLAST.

“Affinity” refers to the total strength of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen).

Affinity can be measured by common methods known in the art, including, for example, Biacore, radioimmunoassay (RIA) and ELISA.

The affinity of a molecule X for its partner Y can generally be represented by equilibrium dissociation constant (KD), calculated as the ratio k_{off}/kₒₙ (k_{d}/kₐ). See, e.g., Chen, Y., et al., (1999) J. MoI Biol 293:865-881. Low-affinity antibodies generally bind antigen slowly and tend to dissociate readily, whereas high-affinity antibodies generally bind antigen faster and tend to remain bound longer. In one embodiment of the invention, the “dissociation rate (k_{d})” is measured by using surface plasmon resonance assays. An “on-rate” or “rate of association” or “association rate (kₐ)” or “kₒₙ” according to this invention can also be determined with the same surface plasmon resonance technique and calculated using a simple one-to-one Langmuir binding model (BIAcore Evaluation Software) by simultaneous fitting the association and dissociation sensorgram.

The term “EC50”, as used herein, refers to the concentration of an antibody or an antigen-binding fragment thereof, which binds to PD-1 and/or induces a response, either in an in vitro or an in vivo assay, which is 50% of the maximal binding or response, i.e., halfway between the maximal binding or response and the baseline.

The terms “cancer” or “neoplasm” and “tumor” can be used interchangeably in the present invention, referring to a neoplasm or tumor resulting from abnormal uncontrolled growth of cells that makes them pathological to the host organism. In some embodiments, cancer refers to a benign tumor, which has remained localized. In other embodiments, cancer refers to a malignant tumor, which has invaded and destroyed neighboring body structures and spread to distant sites. In some embodiments, the cancer is associated with a specific cancer antigen.

As used herein, “treating” or “treatment” of a disease in a subject refers to an approach for obtaining beneficial or desired results, including one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

A “pharmaceutically acceptable carrier” is a carrier with which an active ingredient constitutes a pharmaceutical formulation. A pharmaceutically acceptable carrier includes, but is not limited to, a buffer, excipient, stabilizer, or preservative.

The term “package insert” is used to refer to instructions customarily included in commercial package of a therapeutic product. Generally, there is information about the use of the therapeutic product on the package insert such as indications, usage, dosage, administration, combination therapy, contraindications and/or warnings.

The present invention will be described with respect to particular embodiments and with reference to certain drawings, but the invention is not limited thereto but only by the claims. The term “comprising” as used in the present description and claims does not exclude other elements or steps. Where an indefinite or definite article is used when referring to a singular noun e.g. “a” or “an”, “the”, this includes a plural of that noun unless something else is specifically stated.

### 2. Anti-PD-1 Antibody and Methods of Preparing the Same

This invention encompasses isolated anti-PD-1 antibodies or fragments thereof, polynucleotides comprising sequences encoding the anti-PD-1 antibodies or fragments thereof.

The isolated anti-PD-1 antibodies or fragments thereof bind to PD-1 molecule expressed on cells (for example, cancer cells) with high affinity and facilitating effective immune response against cancer cells. The antibody and its immunoreactive fragments provided herein are capable of enhancing the activation of the immune system, and thus providing important therapeutic and diagnostic agents for use in targeting pathological conditions associated with expression and/or activity of PD-1 molecule. In one aspect, the present invention provides an isolated antibody or antigen binding fragment thereof, comprising a heavy chain (HC) variable region sequence and a light chain (LC) variable region sequence, wherein the antibody binds to an extracellular domain of PD-1 with a binding affinity of better than 10 nM or about 10 nM, better than 8 nM or about 8 nM, better than 6 nM or about 6 nM, better than 4 nM or about 4 nM, better than 2 nM or about 2 nM, better than 1 nM or about nM, as determined by SPR analysis, for example, about 0.5-4 nM, about 0.8-4.0 nM, about 1.0-4.0 nM, about 2.0-4.0 nM, about 3.0-4.0 nM, about 0.6-3.5 nM, about 1.4-3.5 nM, about 2.5-3.5 nM, about 0.7-2.5 nM, about 0.8-2.0 nM, about 1.0-2.0 nM, about 0.4 nM, 0.3 nM, 0.2 nM, 0.1 nM, or better, as determined by SPR analysis.

In certain embodiments, the present invention provides an antibody or antigen binding fragment thereof, comprising at least one of the following:
(a) CDR1H sequence comprising the amino acid sequence as shown by GFTFSSYGMS (SEQ ID NO: 1),
(b) CDR2H sequence comprising the amino acid sequence as shown by IISGGGRDIYYLDSVKG (SEQ ID NO: 2),
(c) CDR3H sequence comprising the amino acid sequence as shown by PIYDAYSFAY (SEQ ID NO: 3),
(d) CDR1L sequence comprising the amino acid sequence as shown by RASQTISNNLH (SEQ ID NO: 4),
(e) CDR2L sequence comprising the amino acid sequence as shown by YASQSIS (SEQ ID NO: 5), and
(f) CDR3L sequence comprising the amino acid sequence as shown by QQSYSWPLT (SEQ ID NO: 6).

In certain embodiments, the present invention provides an antibody or antigen binding fragment thereof, wherein
(a) the HC comprises
CDR1H sequence comprising the amino acid sequence as shown by GFTFSSYGMS (SEQ ID NO: 1),
CDR2H sequence comprising the amino acid sequence as shown by IISGGGRDIYYLDSVKG (SEQ ID NO: 2), and
CDR3H sequence comprising the amino acid sequence as shown by PIYDAYSFAY (SEQ ID NO: 3),
(b) the LC comprises
CDR1L sequence comprising the amino acid sequence as shown by RASQTISNNLH (SEQ ID NO: 4),
CDR2L sequence comprising the amino acid sequence as shown by YASQSIS (SEQ ID NO: 5), and
CDR3L sequence comprising the amino acid sequence as shown by QQSYSWPLT (SEQ ID NO: 6).

In certain embodiments, the antibody is a chimeric antibody, humanized antibody, or human antibody. In certain embodiments, the antibody or antigen binding fragment thereof of the present invention further comprises a human acceptor framework. In certain embodiments, the human acceptor framework is derived from a human immunoglobulin framework or a human consensus framework. In certain embodiments, the human acceptor framework comprises subgroup kappa I framework sequences for VL, and subgroup III framework sequences for VH. Generally, the subgroup of sequences is a subgroup as in Kabat et al., Sequences of Proteins of Immunological Interest, Fifth Edition, NIH Publication 91-3242, Bethesda MD (1991), vols. 1-3. In certain embodiments, for the VL, the subgroup is subgroup kappa I as in Kabat et al, supra. In certain embodiments, for the VH, the subgroup is subgroup III as in Kabat et al, supra.

In certain embodiments, the antibody or antigen binding fragment thereof comprises human consensus framework. In some embodiments, the antibody or antigen binding fragment thereof comprises human consensus framework with amino acid sequence changes, for example, 1-15, 1-10, 2-9, 3-8, 4-7 or 5-6 amino acid changes.

In certain embodiments, the antibody or antigen binding fragment thereof of the invention comprises the HC variable region sequence comprising an amino acid sequence as shown by SEQ ID NO: 7 or 8, or an amino acid sequence having more than 80%, 85%, 90%, 91%, 92%, 93%, 94%, 95%, 96%, 97%, 98% or 99% identity with SEQ ID NO: 7 or 8. In certain embodiments, the antibody or antigen binding fragment thereof of the invention comprises the LC variable region sequence comprising an amino acid sequence as shown by SEQ ID NO: 9 or SEQ ID NO: 10, or an amino acid sequence having more than 80%, 85%, 90%, 91%, 92%, 93%, 94%, 95%, 96%, 97%, 98% or 99% identity with SEQ ID NO: 9 or 10.

In certain embodiments, the HC variable region sequence comprises an amino acid sequence as shown by SEQ ID NO: 7 and the LC variable region sequence comprises an amino acid sequence as shown by SEQ ID NO: 9 or SEQ ID NO: 10. In certain embodiments, the HC variable region sequence comprises an amino acid sequence as shown by SEQ ID NO: 8 and the LC variable region sequence comprises an amino acid sequence as shown by SEQ ID NO: 9 or SEQ ID NO: 10. In certain embodiments, the antibody or antigen binding fragment thereof of the invention comprises the HC sequence comprising an amino acid sequence as shown by SEQ ID NO: 11 or 12, or an amino acid sequence having more than 80%, 85%, 90%, 91%, 92%, 93%, 94%, 95%, 96%, 97%, 98% or 99% identity with SEQ ID NO: 11 or 12. In certain embodiments, the antibody or antigen binding fragment thereof of the invention comprises the LC sequence comprising an amino acid sequence as shown by SEQ ID NO: 13 or SEQ ID NO: 14, or an amino acid sequence having more than 80%, 85%, 90%, 91%, 92%, 93%, 94%, 95%, 96%, 97%, 98% or 99% identity with SEQ ID NO: 13 or 14. In certain embodiments, the HC sequence comprises an amino acid sequence as shown by SEQ ID NO: 11 and the LC sequence comprises an amino acid sequence as shown by SEQ ID NO: 13 or SEQ ID NO: 14. In certain embodiments, the HC sequence comprises an amino acid sequence as shown by SEQ ID NO: 12 and the LC variable region sequence comprises an amino acid sequence as shown by SEQ ID NO: 13 or SEQ ID NO: 14.

In certain embodiments, the antibody is an IgG isotype, for example, IgG1, IgG2 or IgG4 isotype. In certain embodiments, the antigen binding fragment is selected from the group consisting of Fab, F(ab′)2, Fab′, scFv, and Fv. In certain embodiments, the antibody or antigen binding fragment thereof of the invention is a blocking antibody or an antagonist antibody which inhibits or reduces biological activity of the PD-1 molecule it binds. Preferred the blocking antibodies or antagonist antibodies substantially or completely inhibit the biological activity of the PD-1 molecule.

The anti-PD-1 antibodies of the invention are preferably monoclonal. Also encompassed within the scope of the invention are Fab, Fab′, Fab′-SH and F(ab′)2 fragments of the anti-PD-1 antibodies provided herein. These antibody fragments can be created by traditional means, such as enzymatic digestion, or may be generated by recombinant techniques. The anti-PD-1 antibodies and fragments thereof are useful for the diagnostic and therapeutic purposes, including diagnosis and therapy of cancers.

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of different antibodies. The monoclonal anti-PD-1 antibodies of the invention can be made using the hybridoma method or recombinant DNA methods (U.S. Pat. No. 4,816,567).

In the hybridoma method, a mouse or other appropriate host animal, such as a hamster, is immunized by a whole PD-1 molecule or part of the molecule, for example, a polypeptide comprising the extracellular domain of PD-1, together with an adjuvant. A PD-1 molecule or a polypeptide comprising the extracellular domain of A PD-1 molecule may be prepared using methods well-known in the art. In one embodiment, animals are immunized with a polypeptide that contains the extracellular domain (ECD) of PD-1 fused to the Fc portion of an immunoglobulin heavy chain. In one embodiment, animals are immunized with an PD-1-IgG1 fusion protein. Two weeks later the animals are boosted. 7 to 14 days later, animals are bled and the serum is assayed for anti-PD-1 titer. Animals are boosted until titer plateaus.

Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as SP-2 or X63-Ag8-653 cells. Human myeloma and mouse-human heteromyeloma cell lines also have been described for the production of human monoclonal antibodies (Kozbor, J. Immunol, 133:3001 (1984); Brodeur et al, Monoclonal Antibody Production Techniques and Applications, pp. 51-63 (Marcel Dekker, Inc., New York, 1987)).

Culture medium in which hybridoma cells are growing is assayed for production of monoclonal antibodies directed against PD-1. Preferably, the binding specificity of monoclonal antibodies produced by hybridoma cells is determined by immunoprecipitation or by an in vitro binding assay, such as radioimmunoassay (RIA) or enzyme-linked immunoadsorbent assay (ELISA).

The binding affinity of the monoclonal antibody can then be determined by conventional methods in the art. After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)).

Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures.

The anti-PD-1 antibodies of the invention can be made by using combinatorial libraries to screen for synthetic antibody clones with the desired activity or activities. In principle, synthetic antibody clones are selected by screening phage libraries containing phage that display various fragments of antibody variable region (Fv) fused to phage coat protein. Such phage libraries are panned by affinity chromatography against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution. Any of the anti-PD-1 antibodies of the invention can be obtained by designing a suitable antigen screening procedure to select for the phage clone of interest followed by construction of a full length anti-PD-1 antibody clone using the Fv sequences from the phage clone of interest and suitable constant region (Fc) sequences described in Kabat et al, Sequences of Proteins of Immunological Interest, Fifth Edition, NIH Publication 91-3242, Bethesda MD (1991), vols. 1-3.

Repertoires of VH and VL genes can be separately cloned by polymerase chain reaction (PCR) and recombined randomly in phage libraries, which can then be searched for antigen-binding clones as described in Winter et al, Ann. Rev. Immunol, 12: 433-455 (1994). Libraries from immunized sources provide high-affinity antibodies to the immunogen without the requirement of constructing hybridomas. Alternatively, the naive repertoire can be cloned to provide a single source of human antibodies to a wide range of non-self and also self-antigens without any immunization as described by Griffiths et al, EMBO J, 12: 725-734 (1993). Finally, naive libraries can also be made synthetically by cloning the unrearranged V-gene segments from stem cells, and using PCR primers containing random sequence to encode the highly variable CDR3 regions and to accomplish rearrangement in vitro as described by Hoogenboom and Winter, J. MoI Biol, 227: 381-388 (1992).

The antibodies produced by naive libraries (either natural or synthetic) can be of moderate affinity, but affinity maturation can also be mimicked in vitro by constructing and reselecting from secondary libraries. For example, mutation can be introduced at random in vitro by using error-prone polymerase (reported in Leung et al., Technique, 1: 11-15 (1989)) in the method of Hawkins et al., J. MoL Biol., 226: 889-896 (1992) or in the method of Gram et al., Proc. Natl. Acad. Sci USA, 89: 3576-3580 (1992). Additionally, affinity maturation can be performed by randomly mutating one or more CDRs, e.g. using PCR with primers carrying random sequence spanning the CDR of interest, in selected individual Fv clones and screening for higher affinity clones. Another effective approach is to recombine the VH or VL domains selected by phage display with repertoires of naturally occurring V domain variants obtained from unimmunized donors and screen for higher affinity in several rounds of chain reshuffling as described in Marks et al., Biotechnol, 10: 779-783 (1992).

It is possible to select between phage antibodies of different affinities, even with affinities that differ slightly, for PD-1. However, random mutation of a selected antibody (e.g. as performed in some of the affinity maturation techniques described above) is likely to give rise to many mutants, most binding to antigen, and a few with higher affinity. To retain all the higher affinity mutants, phages can be incubated with excess biotinylated PD-1, but with the biotinylated PD-1 at a concentration of lower molarity than the target molar affinity constant for PD-1. The high affinity-binding phages can then be captured by streptavidin-coated paramagnetic beads. Such “equilibrium capture” allows the antibodies to be selected according to their affinities of binding, with sensitivity that permits isolation of mutant clones with as little as two-fold higher affinity from a great excess of phages with lower affinity.

Anti-PD-1 clones may be selected based on performance of activity. In one embodiment, the invention provides anti-PD-1 antibodies that block the binding between an PD-1 receptor and its ligand. Anti-PD-1 antibodies of the invention possessing the properties described herein can be obtained by screening anti-PD-1 hybridoma clones for the desired properties by any convenient method. For example, if an anti-PD-1 monoclonal antibody that blocks or does not block the binding of PD-1 receptor to PD-1 ligand is desired, the candidate antibody can be tested in a binding competition assay, such as a competitive binding ELISA, wherein plate wells are coated with PD-1, and a solution of antibody in an excess of PD-1 receptor is layered onto the coated plates, and bound antibody is detected enzymatically, e.g. contacting the bound antibody with HRP-conjugated anti-Ig antibody or biotinylated anti-Ig antibody and developing the HRP color reaction, e.g. by developing plates with streptavidin-HRP and/or hydrogen peroxide and detecting the HRP color reaction by spectrophotometry at 490 nm with an ELISA plate reader.

### 3. Isolated Polynucleotides, Vectors, Host Cells and Recombinant Methods

Provided herein are isolated polynucleotides, vectors, or host cells comprising the coding sequence of the anti-PD-1 antibodies or fragments thereof of the present disclosure described above. In some embodiments, the anti-PD-1 antibody is the hybridoma-derived monoclonal antibodies or phage display Fv clones of the invention. In some embodiments, hybridoma-derived monoclonal antibodies or phage display Fv clones of the invention is readily isolated and sequenced using conventional procedures (e.g. by using oligonucleotide primers designed to specifically amplify the heavy and light chain coding regions of interest from hybridoma or phage DNA template). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of the desired monoclonal antibodies in the recombinant host cells.

DNA encoding the Fv clones of the invention can be combined with known DNA sequences encoding heavy chain and/or light chain constant regions (e.g. the appropriate DNA sequences can be obtained from Kabat et al, supra) to form clones encoding full or partial length heavy and/or light chains. It will be appreciated that constant regions of any isotype can be used for this purpose, including IgG, IgM, IgA, IgD, and IgE constant regions, and that such constant regions can be obtained from any human or animal species. A Fv clone derived from the variable domain DNA of one animal (such as human) species and then fused to constant region DNA of another animal species to form coding sequence(s) for “hybrid”, full length heavy chain and/or light chain is included in the definition of “chimeric” and “hybrid” antibody as used herein. In a preferred embodiment, a Fv clone derived from human variable DNA is fused to human constant region DNA to form coding sequence(s) for all human, full or partial length heavy and/or light chains.

DNA encoding anti-PD-1 antibodies derived from a hybridoma of the invention can also be modified, for example, by substituting the coding sequence for human heavy- and light-chain constant domains in place of homologous murine sequences derived from the hybridoma clone (e.g. as in the method of Morrison et al, Proc. Natl Acad. Sci. USA, 81: 6851-6855 (1984)). DNA encoding a hybridoma or Fv clone-derived antibody or fragment can be further modified by covalently joining to the immunoglobulin coding sequence all or part of the coding sequence for a non-immunoglobulin polypeptide. In this manner, “chimeric” or “hybrid” antibodies are prepared that have the binding specificity of the Fv clone or hybridoma clone-derived antibodies of the invention.

For recombinant production of an antibody of the invention, the nucleic acid encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the heavy and light chains of the antibody). Many vectors are available. The choice of vector depends in part on the host cell to be used. Generally, preferred host cells are of either prokaryotic or eukaryotic (generally mammalian) origin. It will be appreciated that constant regions of any isotype can be used for this purpose, including IgG, IgM, IgA, IgD, and IgE constant regions, and that such constant regions can be obtained from any human or animal species.

### 4. Conjugates and Methods for Preparing the Same

Conjugates or immunoconjugate of the anti-PD-1 antibody or fragment thereof of the present invention and one or more other molecules such as toxins, for example a calicheamicin, maytansinoids, dolastatins, aurostatins, a trichothecene, and CC1065, and the derivatives of these toxins that have toxin activity, radioactive isotopes and immunomodulators, are also contemplated herein.

In some embodiments, the conjugate is used for treating T-cell lymphoma, B-cell lymphoma or lymphpocytic Leukemia, comprising an antibody (full length or fragments) of the invention conjugated to one or more maytansinoid molecules. Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Conjugates containing maytansinoids, methods of making same, and their therapeutic use are disclosed, for example, in U.S. Pat. Nos. 5,208,020, 5,416,064 and European Patent EP 0 425 235 B1, the disclosures of which are hereby expressly incorporated by reference. Conjugates of the antibody and maytansinoid may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters. In some embodiments, the conjugate comprises an antibody of the invention conjugated to dolastatins or dolostatinpeptidic analogs and derivatives, the auristatins (U.S. Pat. Nos. 5,635,483; 5,780,588). For selective destruction of the tumor, the antibody may comprise a highly radioactive atom. A variety of radioactive isotopes are available for the production of radioconjugated antibodies. The radio- or other labels may be incorporated in the conjugate in known ways. For example, the peptide may be biosynthesized or may be synthesized by chemical amino acid synthesis using suitable amino acid precursors involving, for example, fluorine-9 in place of hydrogen. “Monoclonal Antibodies in Immunoscintigraphy” (Chatal, CRC Press 1989) describes other methods in detail.

In some embodiments, the conjugate is used for treating T-cell lymphoma, B-cell lymphoma or lymphpocytic Leukemia, comprising an antibody (full length or fragments) of the invention conjugated to one or more immunomodulators, wherein the immunomodulators can work synergistically with the antibody (full length or fragments) to enhance immune response against antigens and abnormal cells including tumor cells. In some embodiments, the immunomodulators are anyone selected from the group consisting of checkpoint inhibitors (such as Atezolizumab, Avelumab, Cemiplimab, Durvalumab, Ipilimumab, Nivolumab, Pembrolizumab), cytokines (such as Aldesleukin, Granulocyte-macrophage colony-stimulating factor, IFNα-2a, IFNα-2B, Pre-IFNα-2B), agonists, and adjuvants (such as Imiquimod or Poly ICLC), or the molecules work the same.

Typically, peptide-based drug moieties can be prepared by forming a peptide bond between two or more amino acids and/or peptide fragments. Such peptide bonds can be prepared, for example, according to the liquid phase synthesis method that is well known in the field of peptide chemistry. The auristatin/dolastatin drug moieties may be prepared according to the methods of: U.S. Pat. Nos. 5,635,483; 5,780,588. See also Doronina (2003) Nat Biotechnol 21(7):778-784.

The present invention further contemplates a conjugate formed between an antibody and a compound with nucleolytic activity (e.g., a ribonuclease or a DNA endonuclease such as a deoxyribonuclease; DNase).

### 5. Antibody Fragments and Methods for Preparing the Same

The present invention encompasses antibody fragments. The antibody fragments are the immunoreactive fragments of the anti-PD-1 antibody of the present disclosure. In certain circumstances there are advantages of using antibody fragments, rather than whole antibodies. The smaller size of the fragments allows for rapid clearance, and may lead to improved access to solid tumors.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (Carter et al., Bio/Technology 10:163-167 (1992)). According to another approach, F(ab′)2 fragments can be isolated directly from recombinant host cell culture. Fab and F(ab′)2 fragment with increased in vivo half-life comprising salvage receptor binding epitope residues are described in U.S. Pat. No. 5,869,046. Other techniques for the production of antibody fragments will be apparent to the skilled practitioner.

In other embodiments, the antibody of choice is a single chain Fv fragment (scFv). See WO 93/16185; U.S. Pat. Nos. 5,571,894; and 5,587,458. Fv and scFv are the only known species with intact combining sites that are devoid of constant regions; thus, they are suitable for reduced nonspecific binding during in vivo use. scFv fusion proteins may be constructed to yield fusion of an effector protein at either the amino or the carboxy terminus of an scFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

### 6. Humanized Antibodies and Human Antibodies

The anti-PD-1 antibodies of the present invention in some embodiments are humanized antibodies. Various methods for humanizing non-human antibodies are known in the art. For example, a humanized antibody can have one or more amino acid residues introduced into it from a source which is non-human. These non-human amino acid residues are often referred to as “import” residues, which are typically taken from an “import” variable domain.

Humanization can be essentially performed following the method of Winter and co-workers (Jones et al (1986) Nature 321:522-525; Riechmann et al (1988) Nature 332:323-327; Verhoeyen et al (1988) Science 239:1534-1536), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies. The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework for the humanized antibody (Sims et al (1993) J. Immunol. 151:2296; Chothia et al. (1987) J. MoI. Biol. 196:901. Another method uses a particular framework derived from the consensus sequence of all human antibodies of a particular subgroup of light or heavy chains.

It is further important that antibodies be humanized with retention of high affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for PD-1, is achieved.

Transgenic animals (e.g. mice) that are also capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al, Nature, 362: 255 (1993); Bruggermann et al, Year in Immunol, 7: 33 (1993).

Gene shuffling can also be used to derive human antibodies from non-human, e.g. rodent, antibodies, where the human antibody has similar affinities and specificities to the starting non-human antibody. According to this method, which is also called “epitope imprinting”, either the heavy or light chain variable region of a non-human antibody fragment obtained by phage display techniques as described above is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone, i.e. the epitope governs (imprints) the choice of the human chain partner. When the process is repeated in order to replace the remaining non-human chain, a human antibody is obtained (see PCT WO 93/06213 published Apr. 1, 1993). Unlike traditional humanization of non-human antibodies by CDR grafting, this technique provides completely human antibodies, which have no FR or CDR residues of non-human origin.

### 7. Bispecific Antibodies and Methods for Preparing the Same

Bispecific antibodies are monoclonal, preferably human or humanized, antibodies that have binding specificities for at least two different antigens. In the present case, one of the binding specificities is for PD-1 and the other is for any other antigen. Exemplary bispecific antibodies may bind to two different epitopes of the PD-1 protein. Bispecific antibodies may also be used to localize cytotoxic agents to cells which express PD-1, in this case, antibodies possess an PD-1-binding arm and an arm which binds the cytotoxic agent.

In some embodiments, the bispecific antibodies possess an PD-1-binding arm which comprising the anti-PD-1 antibody or fragment thereof of the present disclosure and an arm which binds to a tumor antigen or an immune checkpoint protein. In some embodiments the tumor antigen is selected from the group consisting of A33; ADAM-9; ALCAM; BAGE; beta-catenin; CA125; Carboxypeptidase M; CD103; CD19; CD20; CD22; CD23; CD25; CD27; CD28; CD36; CD40/CD154; CD45; CD46; CD5; CD56; CD79a/CD79b; CDK4; CEA; CTLA4; Cytokeratin 8; EGF-R; EphA2; ErbB1; ErbB3; ErbB4; GAGE-1; GAGE-2; GD2/GD3/GM2; HER-2/neu; human papillomavirus-E6; human papillomavirus-E7; JAM-3; KID3; KID31; KSA (17-1A); LUCA-2; MAGE-1; MAGE-3; MART; MUC-1; MUM-1; N-acetylglucosaminyltransferase; Oncostatin M; p15; PIPA; PSA; PSMA; ROR1; TNF-β receptor; TNF-a receptor; TNF-γ receptor; Transferrin Receptor; and VEGF receptor. In some embodiments, the immune checkpoint protein is selected from the group consisting of 2B4; 4-1BB; 4-1BB ligand, B7-1; B7-2; B7H2; B7H3; B7H4; B7H6; BTLA; CD155; CD160; CD19; CD200; CD27; CD27 ligand; CD28; CD40; CD40 ligand; CD47; CD48; CTLA-4; DNAM-1; Galectin-9; GITR; GITR ligand; HVEM; ICOS; ICOS ligand; IDOI; KIR; 3DL3; LAG-3; OX40; OX40 ligand; PD-L1; PD-1; PD-L2; LAG3; PGK; SIRP α; TIM-3; TIGIT; VSIG8.

Bispecific antibodies can be prepared as full length antibodies or antibody fragments (e.g. F(ab′)2 bispecific antibodies). Methods for making bispecific antibodies are known in the art. Traditionally, the recombinant production of bispecific antibodies is based on the co-expression of two immunoglobulin heavy chain-light chain pairs, where the two heavy chains have different specificities. Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. According to a different and more preferred approach, antibody variable domains with the desired binding specificities (antibody-antigen combining sites) are fused to immunoglobulin constant domain sequences.

The fusion preferably is with an immunoglobulin heavy chain constant domain, comprising at least part of the hinge, CH2, and CH3 regions. It is preferred to have the first heavy-chain constant region (CH1), containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific antibody provides for a facile way of separation. This approach is disclosed in WO 94/04690. For further details of generating bispecific antibodies see, for example, Suresh et ah, Methods in Enzymology, 121:210 (1986).

### 8. Pharmaceutical Compositions

Therapeutic formulations comprising the anti-PD-1 antibodies fragments, polynucleotides, vectors, host cells, conjugates or bispecific antibodies of the present disclosure are prepared for storage by mixing the anti-PD-1 antibodies, fragments, polynucleotides, vectors, host cells, conjugates or bispecific antibodies of the present disclosure with the desired degree of purity with optional physiologically acceptable carriers, excipients or stabilizers (Remington: The Science and Practice of Pharmacy 20th edition (2000)), in the form of aqueous solutions, lyophilized or other dried formulations. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants such as TWEEN™ PLURONICS™ or polyethylene glycol (PEG).

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions.

Sustained-release preparations may be prepared. Suitable examples of sustained-release preparations include semipermeable matrices of solid hydrophobic polymers containing the immunoglobulin of the invention, which matrices are in the form of shaped articles, e.g., films, or microcapsule.

### 9. Diagnosis and Treatment Uses of Anti-PD-1 Antibodies

In one aspect, based on specific binding of the anti-PD-1 antibodies or fragments thereof disclosed herein to PD-1, the antibodies of the present invention can be used in detection and quantitation of a PD-1 polypeptide in physiological samples, such as urine, plasma, cellular lysate and biopsy samples. Thus, the anti-PD-1 antibodies disclosed herein can be used diagnostically to monitor PD-1 levels in tissues, e.g., to determine the progression of cancers and/or the efficacy of a given treatment regimen. A skilled person in the art knows that the PD-1 antibodies disclosed herein can be coupled with detectable materials to facilitate the detection. In certain embodiments, the anti-PD-1 antibody or fragment thereof disclosed herein is bound to a solid support to facilitate the detection.

In another aspect, based on the specific binding of the antibodies disclosed herein to PD-1, the antibodies of the present invention can be used in, for example, isolating by affinity chromatography methods or immunoprecipitation methods, analyzing or sorting cells by flow cytometry methods, and detecting a PD-1 polypeptide within fixed tissue samples or cell smear samples by immunohistochemistry, cytology analysis, ELISA, or immunoprecipitation methods.

In certain embodiments, the PD-1 molecule to be detected, quantified or analyzed is human PD-1 protein or fragments thereof. In certain embodiments, the PD-1 protein or fragment thereof is disposed in a solution, such as a lysis solution or a solution containing a sub-cellular fraction of a fractionated cell, or present on surface of PD-1-positive cells, or in complexes containing PD-1 and other cellular components.

The detection method of the present invention can be used to detect expression levels of PD-1 polypeptides in a biological sample in vitro as well as in vivo. In vitro techniques for detection of PD-1 polypeptides include enzyme linked immunosorbent assays (ELISAs), Western blots, flow cytometry, immunoprecipitations, radioimmunoassay, and immunofluorescence (e.g., IHC). Furthermore, in vivo techniques for detection of PD-1 polypeptides include introducing into a subject a labeled anti-PD-1 antibody. By way of example only, the antibody can be labeled with a radioactive marker whose presence and location in a subject can be detected by standard imaging techniques.

Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes or other radioactive agents and fluorescent labels, such as fluorescein and rhodamine, and biotin.

The PD-1 antibodies or fragments thereof disclosed herein can be used as diagnostic reagents for any kind of biological sample. In one aspect, the PD-1 antibodies disclosed herein are useful as diagnostic reagents for human biological samples. PD-1 antibodies can be used to detect PD-1 polypeptides in a variety of standard assay formats. Such formats include immunoprecipitation, Western blotting, ELISA, radioimmunoassay, flow cytometry, IHC and immunometric assays.

The present invention also provides for prognostic (or predictive) uses of the anti-PD-1 antibodies and fragments thereof for determining whether a subject is at risk of developing a medical disease or condition associated with increased PD-1 polypeptide expression or activity (e.g., detection of a precancerous cell). Thus the anti-PD-1 antibodies and fragments thereof disclosed herein can be used for prognostic or predictive purpose to prophylactically treat an individual prior to the onset of a medical disease or condition (for example cancer) characterized by or associated with increased PD-1 polypeptide expression or activity.

Another aspect of the present invention provides methods for determining PD-1 expression in a subject to thereby screen therapeutic or prophylactic compounds for a medical disease or condition (for example cancer) characterized by or associated with increased PD-1 polypeptide expression or activity.

In certain embodiments, the medical disease or condition characterized by or associated with PD-1 polypeptide expression or activity or increased PD-1 polypeptide expression or activity stated above is precancerous condition or cancer. In certain embodiments, the prognostic assays can be utilized to identify a subject having or at risk for developing a cancer. Thus, the present invention provides a method for identifying a disease or condition (for example cancer) associated with increased PD-1 polypeptide expression levels in which a test sample is obtained from a subject and the PD-1 polypeptide detected, wherein the presence of increased levels of PD-1 polypeptides compared to a control sample is predictive for a subject having or at risk of developing a disease or condition (for example cancer) associated with increased PD-1 polypeptide expression levels.

In another aspect, the present invention provides methods for determining whether a subject can be effectively treated with a therapeutic agent for a disorder or condition (for example cancer) associated with increased PD-1 polypeptide expression wherein a biological sample is obtained from the subject and the PD-1 polypeptide is detected using the PD-1 antibody. The expression level of the PD-1 polypeptide in the biological sample obtained from the subject is determined and compared with the PD-1 expression levels found in a biological sample obtained from a subject who is free of the disease. Elevated levels of the PD-1 polypeptide in the sample obtained from the subject suspected of having the disease or condition compared with the sample obtained from the healthy subject is indicative of the PD-1-associated disease or condition (for example cancer) in the subject being tested.

In one aspect, the present invention provides for methods of monitoring the treatment efficacy of agents on the expression of PD-1 polypeptides. Such assays can be applied in drug screening and in clinical trials. For example, the effectiveness of an agent to decrease PD-1 polypeptide levels can be monitored in clinical trials of subjects exhibiting elevated expression of PD-1, e.g., patients diagnosed with cancer. An agent that affects the expression of PD-1 polypeptides can be identified by administering the agent and observing a response. In this way, the expression pattern of the PD-1 polypeptide can serve as a marker, indicative of the physiological response of the subject to the agent.

The foregoing are merely exemplary assays for using the anti-PD-1 antibodies and fragments thereof of the present invention. Other methods now or hereafter developed that use the antibodies or fragments thereof for the determination of PD-1 are included within the scope hereof.

In one aspect, the invention provides methods for treating cancers comprise administering an effective amount of an anti-PD-1 antibody or fragments thereof specifically binding PD-1 to a subject in need of such treatment. The antibodies of the present invention can be used to treat, inhibit, delay progression of, prevent/delay recurrence of, ameliorate, or prevent diseases, disorders or conditions associated with expression and/or activity of one or more antigen molecules including PD-1 molecule, or increased expression and/or activity of one or more antigen molecules including PD-1 molecule.

For treatment use of the anti-PD-1 antibody or fragments thereof of the present invention, the appropriate dosage of an antibody of the invention (when used alone or in combination with other agents will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one or multiple times. Depending on the type and severity of the disease, about 1 μg/kg to 15 mg/kg (e.g. 0.1 mg/kg-10 mg/kg) of antibody is a appropriate dosage for administration to the patient, whether, for example, by one or more separate administrations, or by continuous infusion.

Antibodies of the invention can be used either alone or in combination with other compositions in a therapy. For instance, an antibody of the present invention may be co-administered with another antibody, steroids (such as inhalable, systemic or cutaneous steroids), chemotherapeutic agent(s) (including cocktails of chemotherapeutic agents), other cytotoxic agent(s), anti-angiogenic agent(s), cytokines, and/or growth inhibitory agent(s). Such combined therapies noted above include combined administration (where the two or more agents are included in the same or separate formulations), and separate administration, in which case, the anti-PD-1 antibody or fragment thereof of the present invention can be administrated prior to, during and/or following administration of one or more other agents. The effective amounts of therapeutic agents administered in combination depend on such factors as the type of therapeutic agent to be used and the specific patient being treated. and will generally be at the physician's or veterinarian's discretion.

### 10. Kits and Articles of Manufacture

The present invention provides diagnostic methods for determining the expression level of PD-1. In one particular aspect, the present invention provides kits for determining the expression level of PD-1. The kit comprises the anti-PD-1 antibody or fragment thereof disclosed herein and instructions about how to use the kit, for example, instructions for collecting samples and/or performing the detection, and/or analyzing the results. The kits are useful for detecting the presence of PD-1 polypeptides in a biological sample e.g., any body fluid including, but not limited to, e.g., sputum, serum, plasma, lymph, cystic fluid, urine, stool, cerebrospinal fluid, acitic fluid or blood and including biopsy samples of body tissue. The test samples may also be a tumor cell, a normal cell adjacent to a tumor, a normal cell corresponding to the tumor tissue type, a blood cell, a peripheral blood lymphocyte, or combinations thereof.

In certain embodiments, the kit may further comprise one or more other PD-1 antibodies apart from the anti-PD-1 antibody of the present invention, which are capable of binding a PD-1 polypeptide in a biological sample. The one or more of the PD-1 antibodies may be labeled. In certain embodiments, the kit comprises a first antibody, e.g., attached to a solid support, which binds to a PD-1 polypeptide; and, optionally; 2) a second, different antibody which binds to either the PD-1 polypeptide or the first antibody and is conjugated to a detectable label.

The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions written on a package insert about how to use the kit, for example, instructions for collecting samples and/or performing the detection, and/or analyzing the results.

In another aspect, an article of manufacture containing materials useful for the treatment, prevention and/or diagnosis of the disorders described above is provided by the present invention. The article of manufacture comprises a container and a label or package insert on or associated with the container with written instructions of, for example, indications to be treated, administration regimens and warnings. Suitable containers include, for example, bottles, vials, syringes, etc. The containers may be formed from a variety of materials such as glass or plastic. The container holds a composition comprising the anti-PD-1 antibody or fragment thereof of the present invention, which is by itself or when combined with another composition(s) effective for treating, preventing and/or diagnosing the medical disease or condition characterized by or associated with increased expression and/or activity of one or more molecules including PD-1 polypeptide (e.g., cancers).

The article of manufacture may comprise (a) a first container with a composition contained therein, wherein the composition comprises an antibody of the invention; and (b) a second, third or fourth container with a composition comprising another active ingredient. Additionally, the article of manufacture may further comprise a container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### 11. Treatment Methods

The anti-PD-1 antibodies or fragments thereof of the present invention can be used in certain treatment methods. The present invention further encompasses the antibody-based therapies which involve administering effective amount of the antibody or antigen binding fragment thereof, the bispecific antibody, the polypeptide, the conjugate, the composition, the article of manufacture or kit of the present invention described above to a patient, for example human patient or non-human primate, for treating one or more of the disorders or conditions described herein.

In some embodiments, the patient is a patient with tumor. In some embodiments, the patient is with an infection. In one embodiment, the patient has tumor cells or infected cells with the overexpression of PD-1 ligands, for example PD-L1 and/or PD-L2.

Non-limiting examples of cancers include colorectal cancer, endometrial cancer, esophageal cancer, head and neck cancer, thyroid cancer, leukemia (including acute leukemias (e.g., acute lymphocytic leukemia, acute myelocytic leukemia (including myeloblastic, promyelocytic, myelomonocytic, monocytic, and erythroleukemia) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma. And in some embodiments, the infection is viral, bacterial, fungal, or parasite infection. In some certain embodiments, the infection is HIV infection.

Cellular therapies, and in certain embodiments chimeric antigen receptor (CAR) T-cell therapies, are also provided in the present invention. A suitable T cell can be used, that is put in contact with the anti-PD-1 antibodies or the fragments thereof of the present invention (or alternatively engineered to express the anti-PD-1 antibodies or the binding fragments thereof of the present invention). Upon such contact or engineering, the T cell can then be introduced to a cancer patient in need of a treatment. The cancer patient may have a cancer of any of the types as disclosed herein. The T cell can be, for instance, a tumor-infiltrating T lymphocyte, a CD4+ T cell, a CD8+ T cell, or the combination thereof, without limitation. In some embodiments, the T cell is isolated from the cancer patient. In some embodiments, the T cell is provided by a donor or from a cell bank. When the T cell is isolated from the cancer patient, undesired immune reactions can be minimized. When the T cell is provided by a donor other than the patient him- or herself or from a cell bank, the one or more genes encoding T cell receptor and HLA genes may be knocked out.

A specific dosage and treatment regimen for any particular patient will depend upon a variety of factors, including the anti-PD-1 antibodies or fragments thereof of the present invention used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

In some embodiments, the antibody or antigen binding fragment thereof, the bispecific antibody, the polypeptide, the conjugate, the composition, the article of manufacture or kit of the present invention is administered in combination with an antineoplastic agent, an antiviral agent, antibacterial or antibiotic agent or antifungal agents. Any of these agents known in the art may be administered in the compositions of the current disclosure.

In another embodiment, the antibody or antigen binding fragment thereof, the bispecific antibody, the polypeptide, the conjugate, the composition, the article of manufacture or kit of the present invention is administered in combination with a chemotherapeutic agent. Chemotherapeutic agents that may be administered with the compositions of the disclosure include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In an additional embodiment, the antibody or antigen binding fragment thereof, the bispecific antibody, the polypeptide, the conjugate, the composition, the article of manufacture or kit of the present invention is administered in combination with cytokines, wherein the cytokines include, but are not limited to, IL-2, IL-3, IL-4, IL-5, IL-6, IL-7, IL-10, IL-12, IL-13, IL-15, anti-CD40, CD40L, and TNF-α. In additional embodiments, the compositions of the disclosure are administered in combination with other therapeutic or prophylactic regimens, such as, for example, radiation therapy.

The antibody or antigen binding fragment thereof, the bispecific antibody, the polypeptide, the conjugate, the composition, the article of manufacture or kit of the present invention can be used, in some embodiments, together with an immune checkpoint inhibitor.

Immune checkpoints are molecules in the immune system that either turn up a signal (co-stimulatory molecules) or turn down a signal. Many cancers protect themselves from the immune system by inhibiting the T cell signal. An immune checkpoint inhibitor can help stop such a protective mechanism. An immune checkpoint inhibitor may target any one or more of the following checkpoint molecules, 2B4; 4-1BB; 4-1BB ligand, B7-1; B7-2; B7H2; B7H3; B7H4; B7H6; BTLA; CD155; CD160; CD19; CD200; CD27; CD27 ligand; CD28; CD40; CD40 ligand; CD47; CD 48; CTLA-4; DNAM-1; Galectin-9; GITR; GITR ligand; HVEM; ICOS; ICOS ligand; IDOI; KIR; 3DL3; LAG-3; OX40; OX40 ligand; PD-L1; PD-1; PD-L2; LAG3; PGK; SIRP α; TIM-3; PD-1; VSIG8.

Programmed T cell death 1 (PD-1) is a trans-membrane protein found on the surface of T cells, which, when bound to programmed T cell death ligand 1 (PD-L1) on tumor cells, results in suppression of T cell activity and reduction of T cell-mediated cytotoxicity. Thus, PD-1 and PD-L1 are immune down-regulators or immune checkpoint “off switches”. Example PD-1 inhibitor include, without limitation, nivolumab, (Opdivo) (BMS-936558), pembrolizumab (Keytruda, pidilizumab, AMP-224, MEDIO680 (AMP-514, PDR001, MPDL3280A, MEDI4736, BMS-936559 and MSB0010718C. Programmed death-ligand 1 (PD-L1) also known as cluster of differentiation 274 (CD274) or B7 homolog 1 (B7-H1) is a protein that in humans encoded by the CD274 gene. Nonlimiting examples of PD-L1 inhibitor include Atezolizumab (Tecentriq), Durvalumab (MEDI4736), Avelumab (MSB0010718C), MPDL3280A, BMS935559 (MDX-L105) and AMP-224. CTLA-4 is a protein receptor that downregulates the immune system. Non-limiting examples of CTLA-4 inhibitors include ipilimumab (Yervoy) (also known as BMS-734016, MDX-010, MDX-101) and tremelimumab (formerly ticilimumab, CP-675,206). Lymphocyte-activation gene 3 (LAG-3) is an immune checkpoint receptor on the cell surface works to suppress an immune response by action to Tregs as well as direct effects on CD8+ T cells. LAG-3 inhibitors include, without limitation, LAG525 and BMS-986016. CD28 is constitutively expressed on almost all human CD4+ T cells and on around half of all CD8 T cells. prompts T cell expansion. Non-limiting examples of CD28 inhibitors include TGN1412. CD122 increases the proliferation of CD8+ effector T cells. Non-limiting examples include NKTR-214. 4-IBB (also known as CD137) is involved in T-cell proliferation. CD137-mediated signaling is also known to protect T cells, and in particular CD8+ T cells from activation-induced cell death. PF-05082566, Urelumab (BMS-663513) and lipocalin are example CD137 inhibitors.

For any of the above combination treatments, the antibody or antigen binding fragment thereof, the bispecific antibody, the polypeptide, the conjugate, the composition, the article of manufacture or kit of the present invention can be administered simultaneously or separately from the other anticancer agent.

In one embodiment, a method of treating or inhibiting infection in a patient in need thereof is provided, comprising administering to the patient an effective amount of the antibody or antigen binding fragment thereof, the bispecific antibody, the polypeptide, the conjugate, the composition, the article of manufacture or kit of the present invention.

### EXAMPLES

### Examples 1. Generation of Anti-PD-1 Antibodies

BALB/c mice (6 weeks old, purchased from Beijing Vital River Laboratory Animal Technology Co., Ltd.) were immunized by subcutaneous injection with his-tagged human PD-1 recombinant protein (PD-1/6His, In-house production, NCBI Accession No: NP_005009.2, extracellular domain Pro21-Gln167) with complete Freund adjuvant (SigmaAldrich #F5881). Immunization was repeated 4 times at 3 days intervals. After immunized by protein, mice were immunized 2 times by irradiated human PD-1-expressing Jurkat cells (generated in Example 6). 3 days after final immunization, the Lymph nodes close to the injection site were carefully dissected out. The lymphocytes were fused with P3X63Ag8.653 myeloma cells (Cell Bank, Chinese Academy of Sciences, #TCM10) with PEG1500 (Polyethylene Glycol 1500, Roche #783641, 10×4 mL in 75 mM Hepes, PEG 50% W/V) and cloned with HAT selection (Sigma #H0262) and HFCS (Hybridoma Fusion and Cloning Supplement, 50×, Roche #11-363-735-001). The hybridoma supernatants were screened for the production of antibodies that can bind to human PD-1 by ELISA and a cell-base assay. The selected murine anti-PD-1 clone was humanized using CDR grafting and back mutation.

Antibody humanization by CDR grafting: A selection of acceptor frameworks was made. The variable domain sequences of parental antibody were searched in the database of human germline using NCBI Ig-Blast (http://www.ncbi.nlm.nih.gov/projects/igblast). Five diverse human acceptors (i.e., human variable domains with high homology to the parental antibody) for each heavy chain and light chain were chosen. The CDRs of human acceptors were replaced with their mouse counterparts, resulting in humanized variable domain sequences. Then, back mutation was conducted and 4 heavy chains and light chains were generated. The murine CDR sequences of heavy chain and light chain (SEQ ID NOs: 1-6) are shown below respectively. The coding genes of nine humanized heavy chain and nine humanized light chains with or without back mutation were designed, synthesized and inserted into an expression vector. The humanized antibodies were expressed, and then used for affinity ranking test.

### Examples 2. Expression and Purification of Anti-PD-1 Antibodies

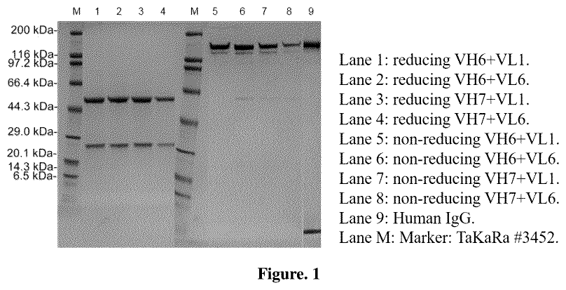

The DNA sequences encoding humanized IgG heavy chain and light chain were synthesized and inserted into pTT5 vector (commercially available from Genscript Biotech Corporation) to construct expression plasmids of full-length IgGs. Expression of chimeric antibody was conducted in HEK293 cell culture (commercially available from ThermoFisher Scientific) and the supernatants were purified with protein A affinity column (Yeasen #36410ES08). The purified antibodies were buffer-exchanged into PBS using PD-10 desalting column (commercially available from ThermoFisher Scientific). The concentration and purity of the purified antibodies were determined by OD280 and SDS-PAGE respectively. The humanized antibodies were expressed in HEK 293 cell culture. The cells were spun down. The supernatants were filtered and conducted with SDS-PAGE analysis (Figure. 1). Random human IgG mixture (commercially available from Genscript Biotech Corporation) was adopted as control. It was shown that the humanized antibodies were successfully expressed and purified.

### Examples 3. SPR Analysis the Binding Affinity of Anti-PD-1 Antibodies to Human PD-1

Anti-human Fc gamma specific antibody (Jackson ImmunoResearch #109-005-098) was immobilized onto the sensor chip using amine coupling method. The humanized antibodies secreted to the culture medium plus the Chimeric VH+VL (Parental mouse VH+VL combined with human Fc) were injected and captured by anti-human Fc antibody via Fc (capture phase) individually. After equilibration, PD-1 was injected for 200 seconds (association phase) followed by the injection of running buffer for 600s (dissociation phase). Responses of reference flow cell (flow cell 1) were subtracted from those of humanized antibodies flow cells during each cycle. The surface was regenerated before the injection of other humanized antibodies. The process was repeated until all antibodies are analyzed. The off-rates of humanized antibodies were obtained from fitting the experimental data locally to 1:1 interaction model using the Biacore 8K evaluation software. The antibodies were ranked by their dissociation rate constants (off-rates, kd). The binders that interact with PD-1 with similar affinity to parental antibody were selected (Table 1).

TABLE 1
Affinity measurement data

| Antibody | Analyte | ka (1/Ms) | kd (1/s) | KD (M) | Rmax | Chi² (RU²) |
|---|---|---|---|---|---|---|
| VH6 + VL1 | PD-1 | 1.13E+05 | 4.04E−04 | 3.57E−09 | 50.2 | 3.41E−02 |
| VH6 + VL6 | PD-1 | 1.56E+05 | 1.27E−04 | 8.12E−10 | 52.3 | 1.45E−01 |
| VH7 + VL1 | PD-1 | 1.04E+05 | 3.17E−04 | 3.04E−09 | 46.4 | 2.88E−01 |
| VH7 + VL6 | PD-1 | 1.54E+05 | 2.18E−04 | 1.41E−09 | 46.1 | 1.14E−01 |
| Chimeric VH + VL | PD-1 | 2.07E+05 | 1.63E−04 | 7.87E−10 | 51.8 | 2.01E−02 |

Accordingly, VH6+VL1, VH6+VL6, VH7+VL1 and VH7+VL6 were selected for further characterization. The sequences of the antibodies or the fragments thereof in table 2 were shown below.

CDR1H amino acid sequence
(SEQ ID NO: 1)
GFTFSSYGMS
CDR2H amino acid sequence
(SEQ ID NO: 2)
IISGGGRDIYYLDSVKG
CDR3H amino acid sequence
(SEQ ID NO: 3)
PIYDAYSFAY
CDR1L amino acid sequence
(SEQ ID NO: 4)
RASQTISNNLH
CDR2L amino acid sequence
(SEQ ID NO: 5)
YASQSIS
CDR3L amino acid sequence
(SEQ ID NO: 6)
QQSYSWPLT
Variable heavy chain domain (VH6) amino acid sequence
(SEQ ID NO: 7)
EVQLVESGGGLVQPGGSLRLSCAASGFTFSSYGMSWVRQAPGKRLEWVAIISG
GGRDIYYLDSVKGRFTISRDNSKNTLYLQMNSLRAEDTAVYYCSSPIYDAYSFAYWG
QGTLVTVSS
Variable heavy chain domain (VH7) amino acid sequence
(SEQ ID NO: 8)
EVQLVESGGGLVQPGGSLRLSCAASGFTFSSYGMSWVRQAPGKGLEWVAIISG
GGRDIYYLDSVKGRFTISRDNSKNNLYLQMNSLRAEDTAVYYCSSPIYDAYSFAYWG
QGTLVTVSS
Variable light chain domain (VL1) amino acid sequence
(SEQ ID NO: 9)
EIVMTQSPATLSVSPGERATLSCRASQTISNNLHWYQQKPGQAPRLLIYYASQSI
SGIPARFSGSGSGTEFTLTISSLQSEDFAVYYCQQSYSWPLTFGGGTKLEIK
Variable light chain domain (VL6) amino acid sequence
(SEQ ID NO: 10)
EIVLTQSPATLSVSPGERATLSCRASQTISNNLHWYHQKPGQAPRLLIKYASQSI
SGIPSRFSGSGSGTDFTLTISSLQSEDFAVYFCQQSYSWPLTFGGGTKLEIK
Heavy chain amino acid sequence 1 (HC1) comprising VH6
(SEQ ID NO: 11, full length sequence)
EVQLVESGGGLVQPGGSLRLSCAASGFTFSSYGMSWVRQAPGKRLEWVAIISG
GGRDIYYLDSVKGRFTISRDNSKNTLYLQMNSLRAEDTAVYYCSSPIYDAYSFAYWG
QGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTH
TCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK
Heavy chain amino acid sequence 2 (HC2) comprising VH7
(SEQ ID NO: 12, full length sequence)
EVQLVESGGGLVQPGGSLRLSCAASGFTFSSYGMSWVRQAPGKGLEWVAIISG
GGRDIYYLDSVKGRFTISRDNSKNNLYLQMNSLRAEDTAVYYCSSPIYDAYSFAYWG
QGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTH
TCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK
Light chain amino acid sequence 1 (LC1) comprising VL1
(SEQ ID NO: 13, full length sequence)
EIVMTQSPATLSVSPGERATLSCRASQTISNNLHWYQQKPGQAPRLLIYYASQSI
SGIPARFSGSGSGTEFTLTISSLQSEDFAVYYCQQSYSWPLTFGGGTKLEIKRTVAAPS
VFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
Light chain amino acid sequence 2 (LC2) comprising VL6
(SEQ ID NO: 14, full length sequence)
EIVLTQSPATLSVSPGERATLSCRASQTISNNLHWYHQKPGQAPRLLIKYASQSI
SGIPSRFSGSGSGTDFTLTISSLQSEDFAVYFCQQSYSWPLTFGGGTKLEIKRTVAAPSV
FIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC
Heavy chain amino acid sequence comprising Chimeric VH
(SEQ ID NO: 15, full length sequence)
EVKLVESGGGLVKPGGSLKLSCAASGFTFSSYGMSWVRQTPEKRLEWVAIISG
GGRDIYYLDSVKGRFTISRDNAKNNLYLQMSSLRSEDTAFYYCSSPIYDAYSFAYWG
QGTLVTVSAASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTH
TCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK
Light chain amino acid sequence comprising Chimeric VL
(SEQ ID NO: 16, full length sequence)
DIVLVQSPATLSVTPGDSVSLSCRASQTISNNLHWYHQKSHESPRLLIKYASQSI
SGIPSRFSGSGSGTDFTLSINSVETEDFGMYFCQQSYSWPLTFGAGTNLELKRTVAAPS
VFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC

For further explanation, the inclusion relationships between the above sequences are shown in Table 2. Sequences on the right are comprised in the sequences on the left in the same row.

TABLE 2

| Whole Chain | | Variable region | | CDR | |
|---|---|---|---|---|---|
| Name | Sequence | Name | Sequence | Name | Sequence |
| HC1 | SEQ ID | VH6 | SEQ ID | CDR1H | SEQ ID |
| | NO: 11 | | NO: 7 | | NO: 1 |
| | | | | CDR2H | SEQ ID |
| | | | | | NO: 2 |
| HC2 | SEQ ID | VH7 | SEQ ID | CDR3H | SEQ ID |
| | NO: 12 | | NO: 8 | | NO: 3 |
| LC1 | SEQ ID | VL1 | SEQ ID | CDR1L | SEQ ID |
| | NO: 13 | | NO: 9 | | NO: 4 |
| | | | | CDR2L | SEQ ID |
| | | | | | NO: 5 |
| LC2 | SEQ ID | VL6 | SEQ ID | CDR3L | SEQ ID |
| | NO: 14 | | NO: 10 | | NO: 6 |

### Examples 4. Binding to Human and Cynomolgus PD-1 Measured by ELISA

MaxiSorp 96-well plates (NUNC #449824) were coated with 2 μg/mL human PD-1/His (in-house production) or cynomolgus PD-1/His protein (ACROBiosystems #PD1-C5223) in 1× PBS (50 μL/well). Plates were incubated at 4° C. overnight. Coating solution was removed and plates were washed once with 200 μL/well PBST (1× PBS containing 0.05% tween-20). Then 200 μL/well blocking buffer (1× PBS with 0.05% tween-20, 3% BSA) was added and incubated at room temperature for 1 hour. Blocking buffer was removed and plates were washed with three times with 200 μL/well PBST. Antibody VH7+VL6 (produced in Example 2) and human IgG1 isotype control (hIgG1, Sigma #I5154-1MG) was diluted by 1× PBS and added to plates (50 μL/well). Plates were incubated at room temperature for 2 hours. Antibodies in the wells were removed and plates were washed three times with 200 μL/well PBST. Goat anti-human IgG (H&L)-HRP secondary antibody (Jackson Immuno Research #109-035-088) was diluted 1:5000 in 1× PBS and added to each well (50 μL/well). Plates were incubated at room temperature for 1 hour. Secondary antibody was removed and plates were washed five times with 200 μL/well PBST. 50 μL/well TMB (eBioscience #85-00-4201-56) was added and plates were incubated at room temperature for several minutes. Then 50 μL/well 2N H2SO4 was added to stop the reaction. Optical density was measured at 450 nm.

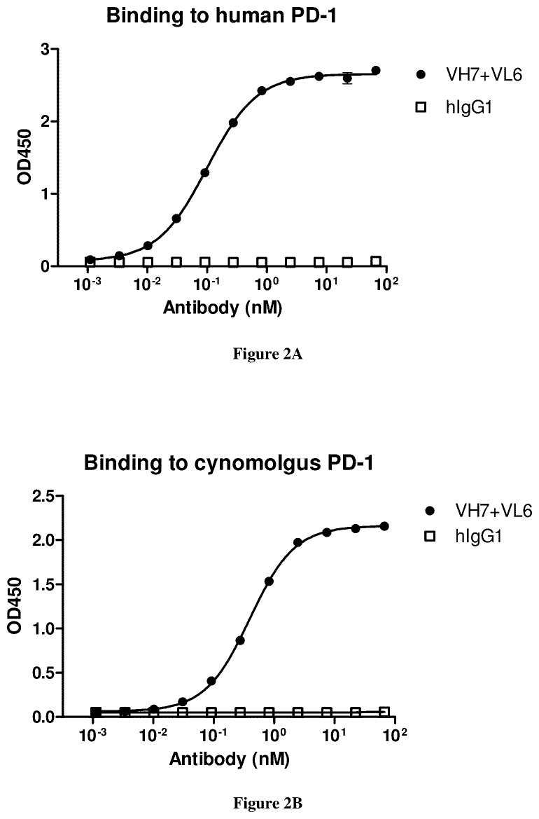

The anti-PD-1 antibody VH7+VL6 bound to human and cynomolgus PD-1 with EC50 of 0.10 nM and 0.40 nM respectively (Table 3). These results indicate that anti-PD-1 antibody can bind to human and cynomolgus PD-1 with high affinity (FIGS. 2A and 2B).

TABLE 3
Binding to human and cynomolgus PD-1
EC50 (nM)

| | Human PD-1 | Cynomolgus PD-1 |
|---|---|---|
| VH7 + VL6 | 0.10 | 0.40 |

### Examples 5. Binding to Human PD-1 on Jurkat Cells

Human PD-1-expressing Jurkat cells (generated in Example 6) were incubated with different concentrations of anti-PD-1 antibody VH7+VL6 or human IgG1 isotype control (Sigma #I5154-1MG) at 4° C. for 30 minutes. Then cells were washed once by FACS buffer (PBS with 2% FBS) and incubated with Alexa Fluor 594 AffiniPure Goat Anti-human IgG secondary antibody (Jackson ImmunoResearch #109-585-088) at 4° C. for 30 minutes. After washed once by FACS buffer, cells were resuspended in 200 μL FACS buffer. The cells after staining were analyzed by BD LSRFortessa Flow Cytometer.

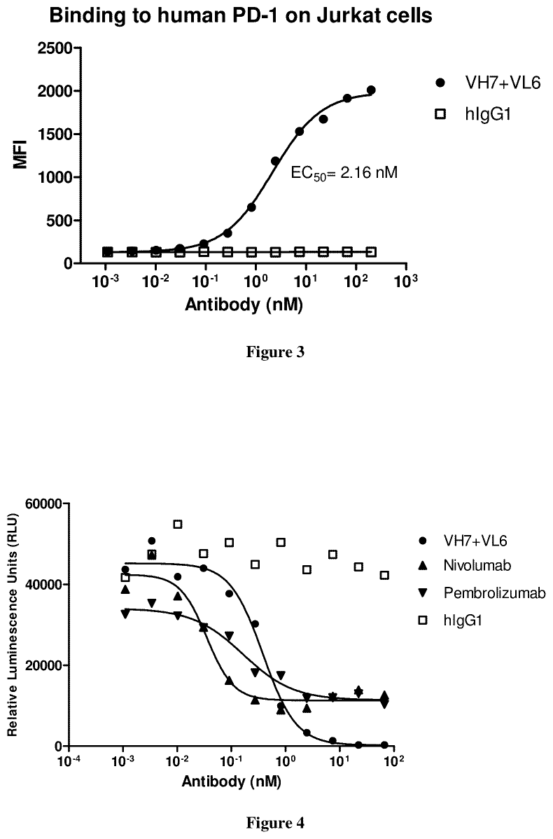

As shown in FIG. 3, anti-PD-1 antibody VH7+VL6 bound to human PD-1 expressed on Jurkat with a EC50 of 2.16 nM.

### Examples 6. Blocking Effect of Anti-PD-1 Antibodies for the Interaction Between PD-1 and PD-L1 in Cell-Based Assay

For this assay, two stable cell lines were generated firstly. A DNA encoding chimeric PD-1 receptor (extracellular and transmembrane domains of human PD-1 were fused to cytoplasmic domain of human CD3ξ chain (NCBI Accession No: NP_932170.1)) and a DNA encoding NFAT-luciferase (amplified from pGL4.30[luc2P/NFAT-RE/Hygro], Sigma #E8481) were cloned into pcDNA3.4 vector (Invitrogen #A14697) and transfected into Jurkat cells (Cell Bank, Chinese Academy of Sciences, #TCHU123) by electroporation. Stable cell line was generated by G418 selection and limiting dilution and named Jurkat/PD-1-CD3z/NF-luci cells. Using the same method, a stable cell line expressing full-length human PD-L1 (NCBI Accession No: NP_054862.1) was generated on CHO-K1 cells (Cell Bank, Chinese Academy of Sciences, #GNHa7), named CHO/PD-L1 cells.

The day before co-culture, CHO/PD-L1 cells were seeded into 96-well flat bottom plate (NUNC #167008) (5×10⁴ cells/well) in complete RPMI1640 medium (Thermo Fisher #C11875500BT) containing 10% FBS (Gibco #16000-044) and 1% Penicillin-Streptomycin (Corning #30-002-CI) and incubated in a CO2 incubator overnight. Jurkat/PD-1-CD3z/NF-luci cells were pre-incubated with anti-PD-1 antibody or human IgG1 isotype control (Sigma #I5154-1MG) for 30 minutes prior to co-culture with CHO/PD-L1 cells. Then removed the culture medium of CHO/PD-L1 cells, seeded the Jurkat/PD-1-CD3z/NF-luci cells with antibody into the wells (1×10⁵ cells/well). After 6 hours, detected fluorescence signals by using the kit of Luciferase Assay System (Promega #E1500).

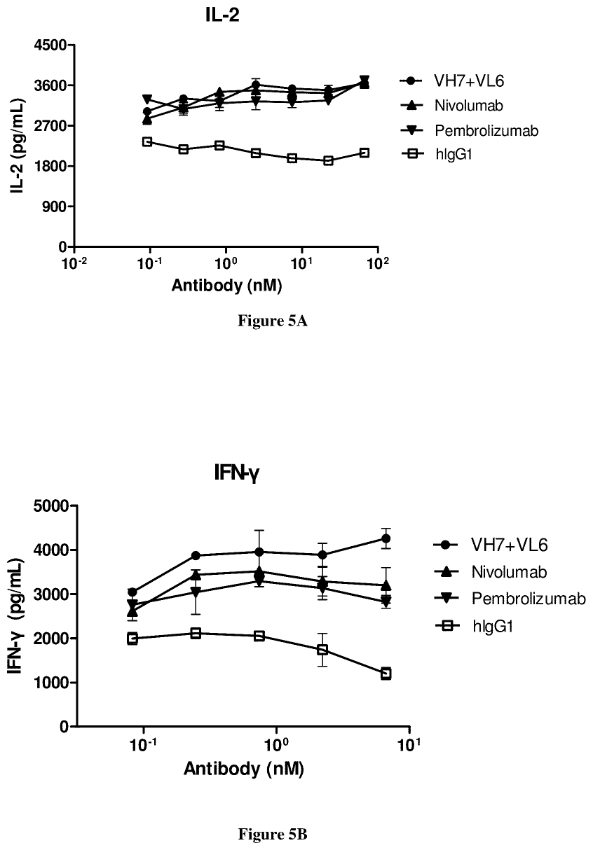

The results are showed in Table 4 and FIG. 4, antibody VH7+VL6 completely inhibited the fluorescence signal induced by CHO/PD-L1 cells, which indicated that antibody VH7+VL6 could block the interaction of PD-1 and PD-L1 efficiently. Nivolumab (CAS #946414-94-4, produced by ChemParter, Shanghai) and Pembrolizumab (CAS #1374853-91-4, produced by ChemParter, Shanghai) inhibited the fluorescence signal by 75% and 71%.

TABLE 4
Inhibition of luciferase signal by anti-PD-1 antibodies

| Antibody | EC50 (nM) | Max inhibition (%) |
|---|---|---|
| VH7 + VL6 | 0.38 | 100 |
| Nivolumab | 0.04 | 75 |
| Pembrolizumab | 0.16 | 71 |

### Examples 7. Enhancement of IL-2, IFN-γ and TNF-α Production on Human PBMCs

### IL-2 Release Assay

96-well flat bottom plate (NUNC #167008) was coated with staphylococcal enterotoxin B (SEB, 0.1 μg/mL in PBS, 100 μL/well) (Provided by Academy of Military Medical Science, China) overnight at 4° C. The next day, human PBMCs isolated from healthy donor were suspended in complete RPMI1640 medium (Thermo Fisher #C11875500BT) containing 10% FBS (Gibco #16000-044) and 1% Penicillin-Streptomycin (Corning #30-002-CI). Then PBMCs were seeded into the pre-coated plate (3×10⁵ cells/well) and incubated with different concentrations of anti-PD-1 antibodies or human IgG1 isotype control (Sigma #I5154-1MG) for 72 hours in a CO2 incubator. Culture supernatants were collected and IL-2 levels were assessed by Human IL-2 DuoSet ELISA Kit (R&D systems #DY202) according to the manufacturer's instructions.

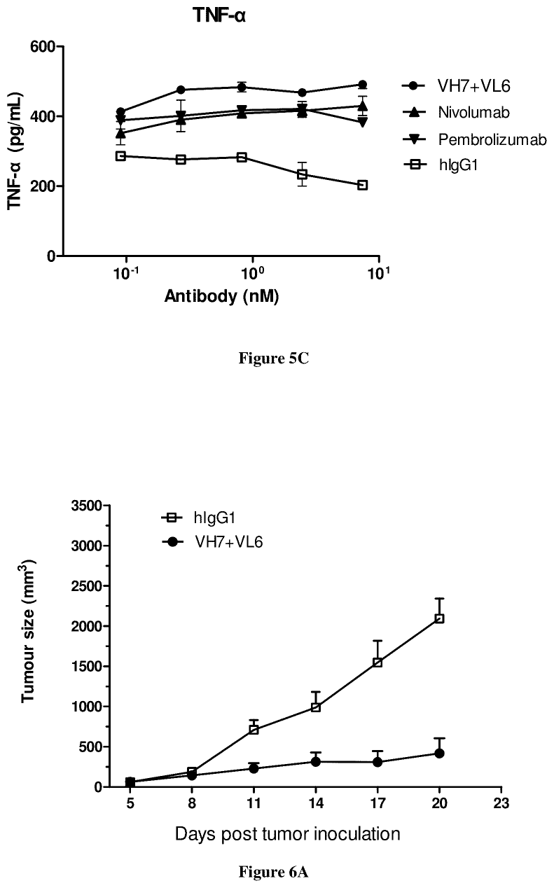

FIG. 5A demonstrated that antibody VH7+VL6, Nivolumab and Pembrolizumab increased IL-2 secretion by PBMCs at equal level.

### IFN-γ and TNF-α Release Assay

100 mm TC-treated Cell Culture Dish (BD Falcon #353003) was coated with SEB (40 ng/mL in 17 mL PBS) overnight at 4° C. The next day, 5×10⁷ cells of human PBMCs were suspended in 20 mL of complete RPMI1640 medium containing 10% FBS and 1% Penicillin-Streptomycin and seeded into the pre-coated dish. Incubated the dish in a CO2 incubator. 96-well flat bottom plate was coated with SEB (4 ng/mL in PBS, 100 μL/well) overnight at 4° C. After 72 hours of incubation, PBMCs were harvested by centrifugation and washed once by complete RPMI1640 medium. Then PBMCs were pre-incubated with different concentrations of anti-PD-1 antibodies or human IgG1 isotype control (Sigma #I5154-1MG) for 30 minutes and seeded into the plate (3×10⁵ cells/well) pre-coated with SEB. After incubation in CO2 incubator for 24 hours, culture supernatants were collected and IFN-γ and TNF-α levels were assessed by Human IFN-γ DuoSet ELISA (R&D systems #DY285B) and Human TNF-α DuoSet ELISA (R&D systems #DY210) kits according to the manufacturer's instructions.

Consistent to the result of IL-2 release assay, all anti-PD-1 antibodies increased IFN-γ and TNF-α secretions obviously. The increased production of both IFN-γ and TNF-α trigged by antibody VH7+VL6 was higher than that trigged by Nivolumab and Pembrolizumab (FIGS. 5B and 5C).

### Examples 8. In Vivo Animal Study of Anti-Tumor Activity

### Antibody Expression and Purification for Animal Study

The DNA sequences encoding VH7 (SEQ ID NO: 8) and VL6 (SEQ ID NO: 10) were subcloned into pcDNA3.4 vector (Invitrogen #A14697) to construct two plasmids, pcDNA3.4-VH7 and pcDNA3.4-VL6. pcDNA3.4-VH7 and pcDNA3.4-VL6 were prepared by using endotoxin-free Plasmid DNA Maxiprep Kit (TIANGEN #DP117). Antibody expression was conducted in 293-F (Invitrogen #R79007). Antibody in the culture supernatant was purified by protein A affinity column (Yeasen #36410ES08). The purified antibody was buffer-exchanged into Histidine buffer (20 mM Histidine, 5% Sucrose, 0.02% Tween 80, pH5.5) by dialysis. The concentration and purity of the purified antibody were determined by OD280 and SDS-PAGE respectively.

### Animal Study

In this study, a CT26-bearing human PD-1 knockin mouse tumor model was used for investigating the anti-tumor activity of antibody VH7+VL6.

Mouse colon carcinoma cells CT26 (Cell Bank, Chinese Academy of Sciences, #TCM37) were cultured in RPMI1640 medium with 10% FBS and 1% Penicillin-Streptomycin. 5×10⁵ CT26 cells in 100 μL of PBS were injected subcutaneously at right dorsal flank of each human PD-1 knockin mouse (BALB/c, female, 6-8 weeks old, GemPharmatech). When the average tumor volume reached about 63 mm3, mice were randomly grouped with 8 mice per group and antibodies were administered. Anti-PD-1 antibody VH7+VL6 was injected intraperitoneally at a dose of 5 mg/kg on days 5, 8, 11, 14 and 17. Mice in control group were injected with human IgG1 isotype control (Bioxcell #BP0085). Tumors were measured every two days using a caliper. Tumor volumes were calculated according to the following formula: width²×length/2 (mm³). Mice were euthanized when the average tumor volume of any group reached 2000 mm3.

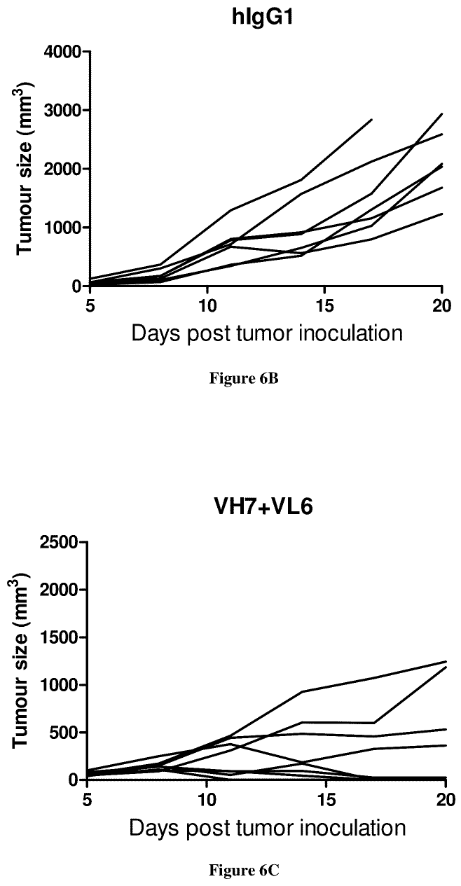

Results showed in FIG. 6A demonstrated that antibody VH7+VL6 strongly inhibited tumor growth in vivo and 50% of tumors were completely regressed at day 20 (FIGS. 6B and 6C). In the control group that mice treated with human IgG1 isotype control, tumors grew much faster and larger. There was no significant change in body weight associated with administration of antibodies.

1. An isolated antibody or antigen binding fragment thereof, comprising a heavy chain (HC) variable region sequence and a light chain (LC) variable region sequence, wherein the antibody binds to an extracellular domain of PD-1 with a binding affinity better than 10 nM as determined by SPR analysis, wherein
(a) the HC comprises
CDR1H sequence comprising the amino acid sequence as shown by GFTFSSYGMS (SEQ ID NO: 1),
CDR2H sequence comprising the amino acid sequence as shown by IISGGGRDIYYLDSVKG (SEQ ID NO: 2), and
CDR3H sequence comprising the amino acid sequence as shown by PIYDAYSFAY (SEQ ID NO: 3),
(b) the LC comprises
CDR1L sequence comprising the amino acid sequence as shown by RASQTISNNLH (SEQ ID NO: 4),
CDR2L sequence comprising the amino acid sequence as shown by YASQSIS (SEQ ID NO: 5), and
CDR3L sequence comprising the amino acid sequence as shown by QQSYSWPLT (SEQ ID NO: 6).

2. The antibody or antigen binding fragment of claim 1, wherein the antibody is a chimeric antibody, humanized antibody, or human antibody.

3. The antibody or antigen binding fragment of claim 1, further comprising a human acceptor framework.

4. The antibody or antigen binding fragment of claim 1, wherein the HC variable region sequence comprises an amino acid sequence as shown by SEQ ID NO: 7 or an amino acid sequence having more than 80%, 85%, 90%, 91%, 92%, 93%, 94%, 95%, 96%, 97%, 98% or 99% identity with SEQ ID NO: 7 or an amino acid sequence as shown by SEQ ID NO: 8 or an amino acid sequence having more than 80%, 85%, 90%, 91%, 92%, 93%, 94%, 95%, 96%, 97%, 98% or 99% identity with SEQ ID NO: 8.

5. (canceled)

6. The antibody or antigen binding fragment of claim 4, wherein the LC variable region sequence comprises an amino acid sequence as shown by SEQ ID NO: 9 or an amino acid sequence having more than 80%, 85%, 90%, 91%, 92%, 93%, 94%, 95%, 96%, 97%, 98% or 99% identity with SEQ ID NO: 9.

7. The antibody or antigen binding fragment of claim 4, wherein the LC variable region sequence comprises an amino acid sequence as shown by SEQ ID NO: 10 or an amino acid sequence having more than 80%, 85%, 90%, 91%, 92%, 93%, 94%, 95%, 96%, 97%, 98% or 99% identity with SEQ ID NO: 10.

8. The antibody or antigen binding fragment thereof of claim 1, wherein the HC variable region sequence comprises an amino acid sequence as shown by SEQ ID NO: 7 or SEQ ID NO: 8 or an amino acid sequence having more than 80%, 85%, 90%, 91%, 92%, 93%, 94%, 95%, 96%, 97%, 98% or 99% identity with SEQ ID NO: 7 or 8, and the LC variable region sequence comprises an amino acid sequence as shown by SEQ ID NO: 9, or SEQ ID NO: 10 or an amino acid sequence having more than 80%, 85%, 90%, 91%, 92%, 93%, 94%, 95%, 96%, 97%, 98% or 99% identity with SEQ ID NO: 9 or 10.

9. The antibody or antigen binding fragment thereof of claim 1, wherein
1) the HC variable region sequence comprises an amino acid sequence as shown by SEQ ID NO: 7 or an amino acid sequence having more than 80%, 85%, 90%, 91%, 92%, 93%, 94%, 95%, 96%, 97%, 98% or 99% identity with SEQ ID NO: 7, and the LC variable region sequence comprises an amino acid sequence as shown by SEQ ID NO: 10 or an amino acid sequence having more than 80%, 85%, 90%, 91%, 92%, 93%, 94%, 95%, 96%, 97%, 98% or 99% identity with SEQ ID NO: 10, or
2) the HC variable region sequence comprises an amino acid sequence as shown by SEQ ID NO: 8 or an amino acid sequence having more than 80%, 85%, 90%, 91%, 92%, 93%, 94%, 95%, 96%, 97%, 98% or 99% identity with SEQ ID NO: 8, and the LC variable region sequence comprises an amino acid sequence as shown by SEQ ID NO: 10 or an amino acid sequence having more than 80%, 85%, 90%, 91%, 92%, 93%, 94%, 95%, 96%, 97%, 98% or 99% identity with SEQ ID NO: 10.

10. The antibody or antigen binding fragment thereof of claim 1, wherein the antibody is an IgG isotype.

11. The antibody or antigen binding fragment thereof of claim 1, wherein the antigen binding fragment is selected from the group consisting of Fab, F(ab′)₂, Fab′, scFv, Fv, Fd, dAb, and diabody.

12. The antibody or antigen binding fragment of claim 1, further comprising a second antibody or antigen binding fragment of claim 1, wherein said antibody is bispecific.

13. The antibody or antigen binding fragment of claim 12, wherein the second antibody or antigen binding fragment thereof specifically binds to a tumor antigen expressed on the surface of a tumor cell, wherein the tumor antigen is selected from the group consisting of A33; ADAM-9; ALCAM; BAGE; beta-catenin; CA125; Carboxypeptidase M; CD103; CD19; CD20; CD22; CD23; CD25; CD27; CD28; CD36; CD40/CD154; CD45; CD46; CD5; CD56; CD79a/CD79b; CDK4; CEA; CTLA4; Cytokeratin 8; EGF-R; EphA2; ErbB1; ErbB3; ErbB4; GAGE-1; GAGE-2; GD2/GD3/GM2; HER-2/neu; human papillomavirus-E6; human papillomavirus-E7; JAM-3; KID3; KID31; KSA (17-1A); LUCA-2; MAGE-1; MAGE-3; MART; MUC-1; MUM-1; N-acetylglucosaminyltransferase; Oncostatin M; p15; PIPA; PSA; PSMA; ROR1; TNF-β receptor; TNF-a receptor; TNF-γ receptor; Transferrin Receptor; and VEGF receptor.

14. A conjugate comprising the antibody or antigen binding fragment thereof of claim 1 linked to a therapeutic agent.

15. The conjugate of claim 14, wherein the therapeutic agent is a cytotoxin or a radioactive isotope.

16. A composition comprising the antibody or antigen binding fragment of claim 1, and a bispecific antibody having antigen binding fragment of claim 1 linked to a second antibody or antigen binding fragment of claim 1,
or a conjugate comprising the antibody or antigen binding fragment thereof of claim 1, and a pharmaceutically acceptable excipient.

17. (canceled)

18. An isolated nucleic acid encoding the antibody or antigen binding fragment thereof of claim 1.

19. An expression vector comprising the nucleic acid of claim 18.

20. A method for treatment of a cancer, comprising administrating an effective amount of the antibody or antigen binding fragment thereof of claim 1.

21. The method of claim 20, wherein the cancer is any one selected from the group consisting of: lymphoma, melanoma, colorectal adenocarcinoma, prostate cancer, breast cancer, colon cancer, lung cancer, liver cancer, gastric cancer, and renal clear cell carcinoma.


SEQUENCE LISTING (16 sequences)

SEQ ID NO: 1 - protein, 10 aa, Artificial Sequence
  CDR1H
GFTFSSYGMS

SEQ ID NO: 2 - protein, 17 aa, Artificial Sequence
  CDR2H
IISGGGRDIYYLDSVKG

SEQ ID NO: 3 - protein, 10 aa, Artificial Sequence
  CDR3H
PIYDAYSFAY

SEQ ID NO: 4 - protein, 11 aa, Artificial Sequence
  CDR1L
RASQTISNNLH

SEQ ID NO: 5 - protein, 7 aa, Artificial Sequence
  CDR2L
YASQSIS

SEQ ID NO: 6 - protein, 9 aa, Artificial Sequence
  CDR3L
QQSYSWPLT

SEQ ID NO: 7 - protein, 119 aa, Artificial Sequence
  VH6
EVQLVESGGGLVQPGGSLRLSCAASGFTFSSYGMSWVRQAPGKRLEWVAIISGGGRDIYYLDSVKGRFTISRDNSKNTLYLQMNSLRAEDTAVYYCSSPIYDAYSFAYWGQGTLVTVSS

SEQ ID NO: 8 - protein, 119 aa, Artificial Sequence
  VH7
EVQLVESGGGLVQPGGSLRLSCAASGFTFSSYGMSWVRQAPGKGLEWVAIISGGGRDIYYLDSVKGRFTISRDNSKNNLYLQMNSLRAEDTAVYYCSSPIYDAYSFAYWGQGTLVTVSS

SEQ ID NO: 9 - protein, 107 aa, Artificial Sequence
  VL1
EIVMTQSPATLSVSPGERATLSCRASQTISNNLHWYQQKPGQAPRLLIYYASQSISGIPARFSGSGSGTEFTLTISSLQSEDFAVYYCQQSYSWPLTFGGGTKLEIK

SEQ ID NO: 10 - protein, 107 aa, Artificial Sequence
  VL6
EIVLTQSPATLSVSPGERATLSCRASQTISNNLHWYHQKPGQAPRLLIKYASQSISGIPSRFSGSGSGTDFTLTISSLQSEDFAVYFCQQSYSWPLTFGGGTKLEIK

SEQ ID NO: 11 - protein, 449 aa, Artificial Sequence
  HC1
EVQLVESGGGLVQPGGSLRLSCAASGFTFSSYGMSWVRQAPGKRLEWVAIISGGGRDIYYLDSVKGRFTISRDNSKNTLYLQMNSLRAEDTAVYYCSSPIYDAYSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 449 aa, Artificial Sequence
  HC2
EVQLVESGGGLVQPGGSLRLSCAASGFTFSSYGMSWVRQAPGKGLEWVAIISGGGRDIYYLDSVKGRFTISRDNSKNNLYLQMNSLRAEDTAVYYCSSPIYDAYSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, Artificial Sequence
  LC1
EIVMTQSPATLSVSPGERATLSCRASQTISNNLHWYQQKPGQAPRLLIYYASQSISGIPARFSGSGSGTEFTLTISSLQSEDFAVYYCQQSYSWPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 214 aa, Artificial Sequence
  LC2
EIVLTQSPATLSVSPGERATLSCRASQTISNNLHWYHQKPGQAPRLLIKYASQSISGIPSRFSGSGSGTDFTLTISSLQSEDFAVYFCQQSYSWPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 449 aa, Artificial Sequence
  HC comprising chimeric VH
EVKLVESGGGLVKPGGSLKLSCAASGFTFSSYGMSWVRQTPEKRLEWVAIISGGGRDIYYLDSVKGRFTISRDNAKNNLYLQMSSLRSEDTAFYYCSSPIYDAYSFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 214 aa, Artificial Sequence
  LC comprising chimeric VL
DIVLVQSPATLSVTPGDSVSLSCRASQTISNNLHWYHQKSHESPRLLIKYASQSISGIPSRFSGSGSGTDFTLSINSVETEDFGMYFCQQSYSWPLTFGAGTNLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC